(19) United States
(12) Patent Application Publication
(10) Pub. No.: US 2015/0044098 A1
(43) Pub. Date: Feb. 12, 2015

(54) HYPERSPECTRAL IMAGING SYSTEMS, UNITS, AND METHODS

(71) Applicant: SCANADU INCORPORATED, MOFFETT FIELD, CA (US)

(72) Inventors: Anthony Smart, Costa Mesa, CA (US); Brandon Woolsey, Sunnyvale, CA (US); Aaron Alexander Rowe, San Francisco, CA (US); Ivo Clarysse, San Francisco, CA (US); Walter De Brouwer, Los Altos, CA (US)

(21) Appl. No.: 14/374,518

(22) PCT Filed: Jan. 30, 2013
(86) PCT No.: PCT/US13/23813

§ 371 Date: Jul. 24, 2014

Related U.S. Application Data
(60) Provisional application No. 61/592,198, filed on Jan. 30, 2012.
(60) Provisional application No. 61/592,237, filed on Jan. 30, 2012.
(60) Provisional application No. 61/592,834, filed on Jan. 31, 2012.
(60) Provisional application No. 61/592,695, filed on Jan. 31, 2012.
(60) Provisional application No. 61/607,222, filed on Mar. 6, 2012.
(60) Provisional application No. 61/608,148, filed on Mar. 8, 2012.
(60) Provisional application No. 61/608,294, filed on Mar. 8, 2012.
(60) Provisional application No. 61/608,339, filed on Mar. 8, 2012.
(60) Provisional application No. 61/608,836, filed on Mar. 9, 2012.
(60) Provisional application No. 61/608,710, filed on Mar. 9, 2012.
(60) Provisional application No. 61/608,733, filed on Mar. 9, 2012.
(60) Provisional application No. 61/608,854, filed on Mar. 9, 2012.
(60) Provisional application No. 61/608,856, filed on Mar. 9, 2012.
(60) Provisional application No. 61/608,904, filed on Mar. 9, 2012.
(60) Provisional application No. 61/608,887, filed on Mar. 9, 2012.
(60) Provisional application No. 61/608,923, filed on Mar. 9, 2012.
(60) Provisional application No. 61/608,937, filed on Mar. 9, 2012.

(51) Int. Cl. G01J 3/28; H04N 5/225; G01J 3/02; H04N 5/30

(52) U.S. Cl. CPC G01J 3/2823; H04N 5/30; H04N 5/2256; G01J 3/0267; G01J 2003/2826; G01N 33/483
USPC 422/82.05; 348/77

(57) ABSTRACT

A hyperspectral imaging system, including: at least one hyperspectral imaging unit, including: at least one lens configured to direct light scattered by, reflected by, or transmitted through a target medium to at least one hyperspectral filter arrangement configured to separate the light into discrete spectral bands; an imaging sensor to: receive the discrete spectral bands from the at least one hyperspectral filter arrangement; detect light by a plurality of pixels for each of the spectral bands; and generate electrical signals based at least in part on at least a portion of the light; and at least one image processor in communication with the at least one imaging sensor and configured to generate hyperspectral image data associated with the target medium; and at least one processor configured to determine biological data based at least partially on at least a portion of the hyperspectral image data.

### CROSS REFERENCE TO RELATED APPLICATIONS

This application claims benefit of priority from U.S. Provisional Patent Application Nos. 61/592,834, filed Jan. 31, 2012; 61/592,198, filed Jan. 30, 2012; 61/592,237, filed Jan. 30, 2012; 61/592,695, filed Jan. 31, 2012, 61/607,222, filed Mar. 6, 2012; 61/608,836, filed Mar. 9, 2012; 61/608,148, filed Mar. 8, 2012; 61/608,710, filed Mar. 9, 2012; 61/608,294, filed Mar. 8, 2012; 61/608,339, filed Mar. 8, 2012; 61/608,733, filed Mar. 9, 2012; 61/608,854, filed Mar. 9, 2012; 61/608,856, filed Mar. 9, 2012; 61/608,904, filed Mar. 9, 2012; 61/608,887, filed Mar. 9, 2012; 61/608,923, filed Mar. 9, 2012; and 61/608,937, filed Mar. 9, 2012, all of which are incorporated by reference in their entirety.

### BACKGROUND OF THE INVENTION

1. Field of the Invention

The present invention relates generally to clinical medical diagnostic systems and arrangements, and in particular to a hyperspectral imaging system for use in determining biological data and information, such as biological data and information associated with or directed to a patient or user.

2. Description of the Related Art

Hyperspectral imaging is an imaging modality that gathers continuous information across a vast portion of the electromagnetic spectrum, as opposed to traditional imaging modalities (e.g., a conventional digital otoscope (such as the digital otoscope shown and described in U.S. Publication No. 2008/0051637)), which generate three bands (red, green, blue (RGB)) per image. Accordingly, hyperspectral cameras and sensors and associated image processing systems have the ability to determine unique hyperspectral fingerprints, or “signatures”, known as “spectral signatures,” where each extra-visible wavelength is assigned, and may be displayed as a visible color. For example, in agricultural and geologic applications, these signatures may be specific to plant species or oil materials, respectively. For medical applications, the majority of hyperspectral imaging has been used to assess superficial skin perfusion via differentiation between oxyhemoglobin and deoxyhemoglobin. This application has been implemented in numerous clinical settings, such as tumor identification and wound healing processes.

While conventional hyperspectral imagers are expensive and bulky, recent developments are leading to smaller hyperspectral imaging systems, such as the system shown and described in U.S. patent application Ser. No. 11/642,867 (U.S. Publication No. 2007/0206242). One example of an available hyperspectral camera is the “Spectral Camera HS”, manufactured by Spectral Imaging, Ltd in Oulu, Finland. This camera is configured to capture hyperspectral images in the 380-800 nm and 400-1,000 nm spectral ranges. Based upon the availability of such miniaturized hyperspectral image sensors, they may be used in connection with devices or applications where conventionally monochrome or RGB image sensors are being used.

As is known, a hyperspectral cube (a “hyper-cube”) is a four-dimensional datacube (including free parameter and intensity), which illustrates or depicts the electromagnetic spectrum of a surface across a very broad visible and extra-visible spectral range. Such a hyper-cube is a three-dimensional hyperspectral image data set, which illustrates the electromagnetic spectral content of a two-dimensional image spectral range. The cube has axes of spatial dimension (X), spatial dimension (Y), and wavelength, and it represents a complete possible spectral analysis of a surface. Of course, it is recognized that the third dimension may be time. The cube represents a stacked set of two-dimensional monochrome images, with each image corresponding to the light intensity at a given spectral band. Since a hyperspectral image contains a full spectral profile of each pixel, the image may be used in determining or obtaining useful information and data.

### SUMMARY OF THE INVENTION

Generally, provided are hyperspectral imaging systems, units, and methods that address or overcome various deficiencies and drawbacks associated with existing systems, especially in the area of medical and biological condition detection and diagnosis. Preferably, provided are hyperspectral imaging systems, units, and methods that are effective in determining biological data and information, such as biological data and information associated with or directed to a patient or user.

Accordingly, in one preferred and non-limiting embodiment, provided is a hyperspectral imaging system, including: at least one hyperspectral imaging unit, including: (i) at least one lens configured to direct light scattered by, reflected from, or transmitted through at least a portion of at least one target medium (and/or region of interest or portion thereof) to at least one hyperspectral filter arrangement configured to separate the light into a plurality of discrete spectral bands; (ii) at least one imaging sensor configured to: (a) receive the plurality of discrete spectral bands from the at least one hyperspectral filter arrangement; (b) detect light by a plurality of pixels for each of the plurality of spectral bands; and (c) generate electrical signals based at least in part on at least a portion of the light; and (ii) at least one image processor in communication with the at least one imaging sensor and configured to generate hyperspectral image data associated with the at least one target medium; and at least one processor configured to determine biological data based at least partially on at least a portion of the hyperspectral image data.

In another preferred and non-limiting embodiment, provided is a hyperspectral unit, including: at least one lens configured to direct light scattered by, reflected from, or transmitted through at least a portion of at least one target medium (and/or region of interest or portion thereof) to at least one hyperspectral filter arrangement configured to separate the light into a plurality of discrete spectral bands; at least one imaging sensor configured to: (a) receive the plurality of discrete spectral bands from the at least one hyperspectral filter arrangement; (b) detect light by a plurality of pixels for each of the plurality of spectral bands; and (c) generate electrical signals based at least in part on at least a portion of the light; and at least one image processor in communication with the at least one imaging sensor and configured to generate hyperspectral image data associated with the at least one target medium; and at least one communication interface configured to communicate at least a portion of the hyperspectral image data to the at least one processor, which is configured to determine biological data based at least partially on at least a portion of the hyperspectral image data.

In another preferred and non-limiting embodiment, the hyperspectral unit at least partially includes a hyperspectral camera, which captures multiple images (e.g., in different dimensions and/or temporally) of a surface or medium, and determines certain specified spectral characteristics. In this embodiment, the system is configured to integrate a hyperspectral imaging camera, an optional light source, a computing device or process, with wired and/or wireless connectivity. After a hyperspectral image is taken by hyperspectral imaging camera, the hyperspectral imaging data are processed (e.g., in “real tune”) using software to determine or detect some medical and/or biological condition associated with the imaged surface.

Since, as discussed, a hyperspectral image includes a full spectral profile of each pixel, the image can be used to detect and locate various portions of the surface associated with a spectral signature, which may be associated with or represent a medical and/or biological (e.g., organic) condition or parameter. Accordingly, the hyperspectral imaging system and unit discussed herein enables superior spectral resolution over multi-spectral and conventional RGB imaging technologies, since each pixel of a hyperspectral image contains hyperspectral image data on the intensity of light in narrow spectral band over a wide range of the electromagnetic spectrum (e.g., from 500 nanometer to 1,000 nanometer), extending into extra-visible region of the infrared. Further, in other embodiments, the hyperspectral imaging system and unit of the present invention may be configured to capture an entire two-dimensional image in a single instance, whereas most hyperspectral imagers today can only image a one dimensional subset of the image at a time, and must scan the sensor over the specimen to produce the desired two-dimensional image (known as the “push-broom” technique). Accordingly, this technique holds considerable advantages over the push-broom technique, such as increased speed of data acquisition, lower sensitivity to sensor/specimen movement, and reduced bulkiness of the sensor apparatus.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic view of one embodiment of a hyperspectral imaging system and unit according to the principles of the present invention;

FIG. 2(a) is a front view of another embodiment of a hyperspectral imaging system and unit according to the principles of the present invention;

FIG. 2(b) is the rear view of the hyperspectral imaging system and unit of FIG. 2(a);

FIG. 3 is a schematic view of one embodiment of a hyperspectral imaging unit according to the principles of the present invention;

FIG. 4 is a schematic view of another embodiment of a hyperspectral imaging system and unit according to the principles of the present invention;

FIG. 5 is a schematic view of one embodiment of a hyperspectral imaging system and unit according to the principles of the present invention;

FIG. 6 is a schematic view of a further embodiment of a hyperspectral imaging system and unit according to the principles of the present invention;

FIG. 7 is a perspective view of another embodiment of a hyperspectral imaging system and unit according to the principles of the present invention;

FIG. 8 is a schematic view of a portion of the hyperspectral imaging system and unit of FIG. 7;

FIG. 9 is a schematic view of another portion of the hyperspectral imaging system and unit of FIG. 7;

FIG. 10 is a schematic view of another embodiment of a hyperspectral imaging system and unit according to the principles of the present invention;

FIG. 11 is a schematic view of a further embodiment of a hyperspectral imaging system and unit according to the principles of the present invention;

FIG. 12 is a schematic view of another embodiment of a hyperspectral imaging system and unit according to the principles of the present invention;

FIG. 13 is a chart illustrating hyperspectral profiles of different results for hyperspectral data or information based on one embodiment of a hyperspectral imaging system and unit according to the principles of the present invention;

FIG. 14 is a schematic view of another embodiment of a hyperspectral imaging system and unit according to the principles of the present invention;

FIG. 15 is a schematic view of a further embodiment of the hyperspectral imaging system and unit of FIG. 14;

FIG. 16 is a schematic view of another embodiment of a hyperspectral imaging system and unit according to the principles of the present invention;

FIG. 17 is a schematic view of a still further embodiment of a hyperspectral imaging system and unit according to the principles of the present invention;

FIG. 18 is a schematic view of a further embodiment of hyperspectral data from the hyperspectral imaging system according to the principles of the present invention;

FIG. 19 is a schematic view of one embodiment of hyperspectral data from the hyperspectral imaging system and unit of FIG. 17;

FIG. 20 is a schematic view of another embodiment of hyperspectral data from the hyperspectral imaging system and unit of FIG. 17;

FIG. 21 is a chart of optical density versus wavelength for hyperspectral data from the hyperspectral imaging system and unit of FIG. 17;

FIG. 22 is a schematic view of a further embodiment of a hyperspectral imaging system and unit according to the principles of the present invention;

FIG. 23 is a schematic view of another embodiment of a hyperspectral imaging system and unit according to the principles of the present invention;

FIG. 24 is a schematic view of a still further embodiment of a hyperspectral imaging system and unit according to the principles of the present invention; and

FIG. 25 is a schematic view of a computer and network infrastructure according to the prior art and for use in connection with a hyperspectral imaging system and unit according to the principles of the present invention.

### DETAILED DESCRIPTION OF THE PREFERRED EMBODIMENTS

For purposes of the description hereinafter, the terms “end”, “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that may be wired and/or wireless in nature. Additionally, two units or components may be in communication with each other even though the data transmitted may be modified, processed, and/or routed between the first and second unit or component. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. It will be appreciated that numerous other arrangements are possible. The components or units may be directly connected to each other or may be connected through one or more other devices or components. The various coupling components for the devices can include but are not limited to the Internet, a wireless network, a conventional wire cable, an optical cable or connection through air, water or any other medium that conducts signals, and any other coupling device or medium.

Generally, and in various preferred and non-limiting embodiments, the invention provides systems and methods for acquiring, evaluating, analyzing, processing and/or presenting hyperspectral image data and/or biological data. For example, in certain preferred and non-limiting embodiments, provided are systems and methods for hyperspectral imaging, such as using a hyperspectral imaging unit included or integrated with a portable device, e.g., a smartphone, a PDA, a handheld device, a pad computer, a laptop, and the like. Various aspects of the invention described herein may be applied to any of the particular applications set forth below or in any other type of medical analytical/diagnostic setting. Further, the invention may be applied as a stand-alone method or system, or as part of an integrated medical diagnostic system. It should be understood that different aspects of the invention can be appreciated individually, collectively, or in combination with each other. In addition, image data may include any type or form of visual, video, and/or observable data, including, but not limited to, a discrete image, a sequence of images, one or more images from a video, a video sequence, and the like.

Hereinafter, this invention is described in terms of functional block components, optional selections, and various processing steps. Such functional blocks may be realized by any number of hardware and/or software components configured to perform to specified functions. For example, the invention may employ various integrated circuit components (e.g., memory elements, processing elements, logic elements, lookup tables, and the like), which may carry out a variety of functions under the control of one or more microprocessors or other control devices. Similarly, the software components of this invention may be implemented with any programming or scripting languages such as C, C#, C++, Java, assembler, extensible markup language (XML), extensible stylesheet transformations (XSLT), with the various algorithms being implemented with any combination of data structures, objects, processes, routines, or other programming elements.

Further, it should be noted that this invention may employ any number of conventional techniques for data transmission, signaling, data processing, network control, and the like. In addition, many applications of the present invention could be formulated. The exemplary network disclosed herein may include any system for exchanging data or transacting processes, such as the Internet, an intranet, an extranet, WAN, LAN, satellite or cellular communication networks, and/or the like. The terms “Internet” or “network”, as used herein, may refer to the Internet, any replacement, competitor or successor to the Internet, or any public or private internetwork, intranet or extranet that is based upon open or proprietary protocols. Specific information related to the protocols, standards, and application software used in connection with the Internet may not be discussed herein.

Where required, a system user may interact with the system to complete a transaction via any input device or user interface, such as presses or gestures on a touch-screen, the user or patient actions that cause a change in readings obtained from sensors, keypad presses, and the like. Similarly, this invention could be used with any kind of smartphone (e.g., Apple iPhone, BlackBerry), handheld computer (e.g., Apple iPad) or used with any type of personal computer, network computer, workstation, minicomputer, mainframe or the like running any operating system, such as any version of Android, Linux, Windows, Windows NT, Windows 2000, Windows XP, MacOS, UNIX, Solaris, iOS or the like. The invention could be implemented using one or more of the following communication protocols: TCP/IP, X.25, SNA, AppleTalk, SCSI, NetBIOS, OSI, GSM, or any number of communication protocols. Moreover, the system contemplates the use, sale, or distribution of any goods, services, or information over any network having similar functionality described herein.

A variety of conventional communications media and protocols may be used for the data links. For example, data links may be an Internet Service Provider (ISP) configured to facilitate communications over a local loop as is typically used in connection with standard modem communication, cable modem, dish networks, ISDN, DSL lines, GSM, G4/LTE, WDMCA, Bluetooth, or any wireless communication media.

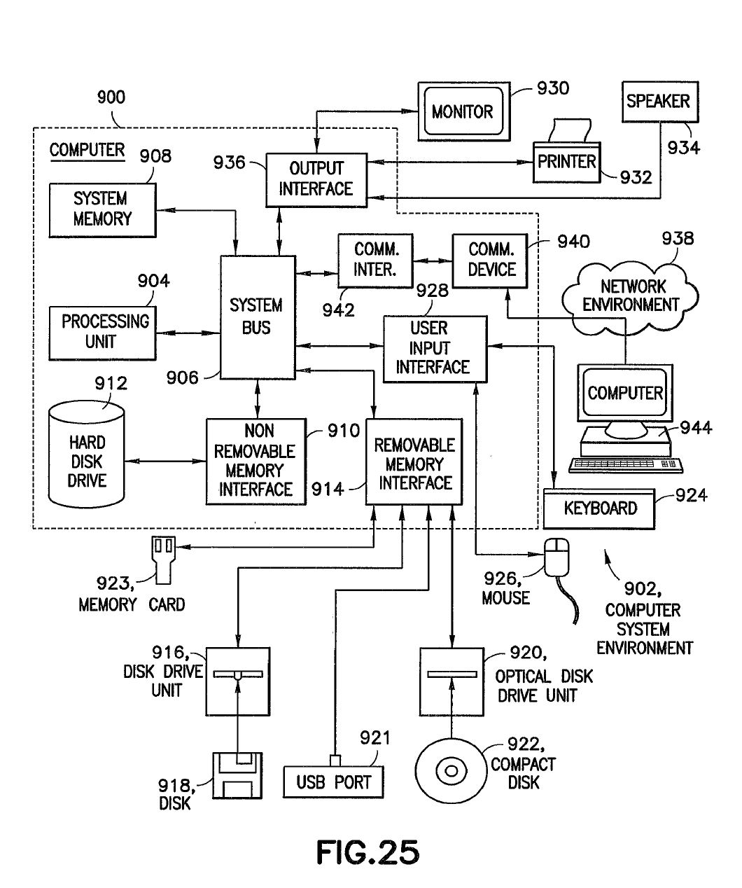

Still further, and discussed hereinafter, it is to be recognized that some or all of the functions, aspects, features, and instances of the present invention may be implemented on a variety of computing devices and systems, wherein these computing devices include the appropriate processing mechanisms and computer-readable media for storing and executing computer-readable instructions, such as programming instructions, code, and the like. As shown in FIG. 25, personal computers 900, 944, in a computing system environment 902 are provided. This computing system environment 902 may include, but is not limited to, at least one computer 900 having certain components for appropriate operation, execution of code, and creation and communication of data. For example, the computer 900 includes a processing unit 904 (typically referred to as a central processing unit or CPU) that serves to execute computer-based instructions received in the appropriate data form and format. Further, this processing unit 904 may be in the form of multiple processors executing code in series, in parallel, or in any other manner for appropriate implementation of the computer-based instructions.

In order to facilitate appropriate data communication and processing information between the various components of the computer 900, a system bus 906 is used. The system bus 906 may be any of several types of bus structures, including a memory bus or memory controller, a peripheral bus, or a local bus using any of a variety of bus architectures. In particular, the system bus 906 facilitates data and information communication between the various components (whether internal or external to the computer 900) through a variety of interfaces, as discussed hereinafter.

The computer 900 may include a variety of discrete computer-readable media components. For example, this computer-readable media may include any media that can be accessed by the computer 900, such as volatile media, non-volatile media, removable media, non-removable media, etc. As a further example, this computer-readable media may include computer storage media, such as media implemented in any method or technology for storage of information, such as computer-readable instructions, data structures, program modules, or other data, random access memory (RAM), read only memory (ROM), electrically erasable programmable read only memory (EEPROM), flash memory, or other memory technology, CD-ROM, digital versatile disks (DVDs), or other optical disk storage, magnetic cassettes, magnetic tape, magnetic disk storage, or other magnetic storage devices, or any other medium which can be used to store the desired information and which can be accessed by the computer 900. Further, this computer-readable media may include communications media, such as computer-readable instructions, data structures, program modules, or other data in other transport mechanisms and include any information delivery media, wired media (such as a wired network and a direct-wired connection), and wireless media. Computer-readable media may include all machine-readable media with the sole exception of transitory, propagating signals. Of course, combinations of any of the above should also be included within the scope of computer-readable media.

The computer 900 further includes a system memory 908 with computer storage media in the form of volatile and non-volatile memory, such as ROM and RAM. A basic input/output system (BIOS) with appropriate computer-based routines assists in transferring information between components within the computer 900 and is normally stored in ROM. The RAM portion of the system memory 908 typically contains data and program modules that are immediately accessible to or presently being operated on by processing unit 904, e.g., an operating system, application programming interfaces, application programs, program modules, program data, and other instruction-based computer-readable codes.

With continued reference to FIG. 25, the computer 900 may also include other removable or non-removable, volatile or non-volatile computer storage media products. For example, the computer 900 may include a non-removable memory interface 910 that communicates with and controls a hard disk drive 912, i.e., a non-removable, non-volatile magnetic medium; and a removable, non-volatile memory interface 914 that communicates with and controls a magnetic disk drive unit 916 (which reads from and writes to a removable, non-volatile magnetic disk 918), an optical disk drive unit 920 (which reads from and writes to a removable, non-volatile optical disk 922, such as a CD ROM), a Universal Serial Bus (USB) port 921 for use in connection with a removable memory card, etc. However, it is envisioned that other removable or non-removable, volatile or non-volatile computer storage media can be used in the exemplary computing system environment 900, including, but not limited to, magnetic tape cassettes, DVDs, digital video tape, solid state RAM, solid state ROM, etc. These various removable or non-removable, volatile or non-volatile magnetic media are in communication with the processing unit 904 and other components of the computer 900 via the system bus 906. The drives and their associated computer storage media discussed above and illustrated in FIG. 25 provide storage of operating systems, computer-readable instructions, application programs, data structures, program modules, program data, and other instruction-based computer-readable code for the computer 900 (whether duplicative or not of this information and data in the system memory 908).

A user may enter commands, information, and data into the computer 900 through certain attachable or operable input devices, such as a keyboard 924, a mouse 926, etc., via a user input interface 928. Of course, a variety of such input devices may be used, e.g., a microphone, a trackball, a joystick, a touchpad, a touch-screen, a scanner, etc., including any arrangement that facilitates the input of data, and information to the computer 900 from an outside source. As discussed, these and other input devices are often connected to the processing unit 904 through the user input interface 928 coupled to the system bus 906, but may be connected by other interface and bus structures, such as a parallel port, game port, or a universal serial bus (USB). Still further, data and information can be presented or provided to a user in an intelligible form or format through certain output devices, such as a monitor 930 (to visually display this information and data in electronic form), a printer 932 (to physically display this information and data in print form), a speaker 934 (to audibly present this information and data), etc. All of these devices are in communication with the computer 900 through an output interface 936 coupled to the system bus 906. It is envisioned that any such peripheral output devices be used to provide information and data to the user.

The computer 900 may operate in a network environment 938 through the use of a communications device 940, which is integral to the computer or remote therefrom. This communications device 940 is operable by and in communication with the other components of the computer 900 through a communications interface 942. Using such an arrangement, the computer 900 may connect with or otherwise communicate with one or more remote computers, such as a remote computer 944, which may be a personal computer, a server, a router, a network personal computer, a peer device, or other common network nodes, and typically includes many or all of the components described above in connection with the computer 900. Using appropriate communication devices 940, e.g., a modem, a network interface or adapter, etc., the computer 900 may operate within and communicate through a local area network (LAN) and a wide area network (WAN), but may also include other networks such as a virtual private network (VPN), an office network, an enterprise network, an intranet, the Internet, etc. It will be appreciated that the network connections shown are exemplary and other means of establishing a communications link between the computers 900, 944 may be used.

As used herein, the computer 900 includes or is operable to execute appropriate custom-designed or conventional software to perform and implement the processing steps of the method and system of the present invention, thereby forming a specialized and particular computing system. Accordingly, the presently-invented method and system may include one or more computers 900 or similar computing devices having a computer-readable storage medium capable of storing computer-readable program code or instructions that cause the processing unit 904 to execute, configure, or otherwise implement the methods, processes, and transformational data manipulations discussed hereinafter in connection with the present invention. Still further, and as discussed, the computer 900 may be in the form of a smartphone, a tablet computer, a personal computer, a personal digital assistant, a portable computer, a laptop, a palmtop, a mobile device, a mobile telephone, a server, or any other type of computing device having the necessary processing hardware to appropriately process data to effectively implement the presently-invented systems, units, and methods.

Computer 944 represents one or more work stations appearing outside the local network and bidders and sellers machines. The bidders and sellers interact with computer 900, which can be an exchange system of logically integrated components including a database server and web server. In addition, secure exchange can take place through the Internet using secure www. An e-mail server can reside on system computer 900 or a component thereof. Electronic data interchanges can be transacted through networks connecting computer 900 and computer 944. Third party vendors represented by computer 944 can connect using EDI or www, but other protocols known to one skilled in the art to connect computers could be used.

The exchange system can be a typical web server running a process to respond to HTTP requests from remote browsers on computer 944. Through HTTP, the exchange system can provide the user interface graphics. It will be apparent to one skilled in the relevant art(s) that the system may utilize databases physically located on one or more computers which may or may not be the same as their respective servers. For example, programming software on computer 900 can control a database physically stored on a separate processor of the network or otherwise.

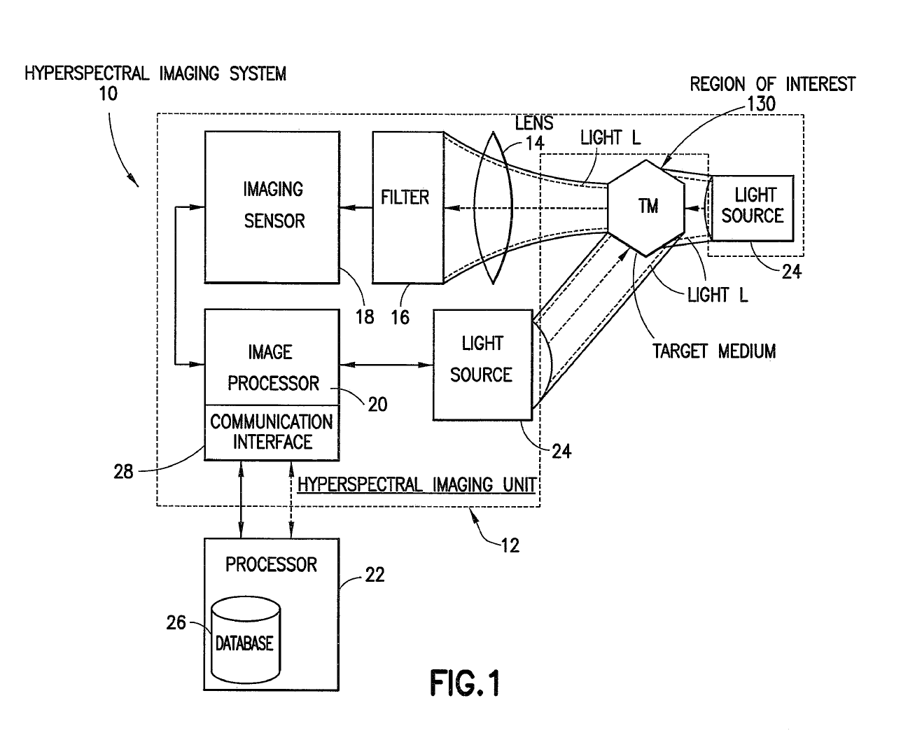

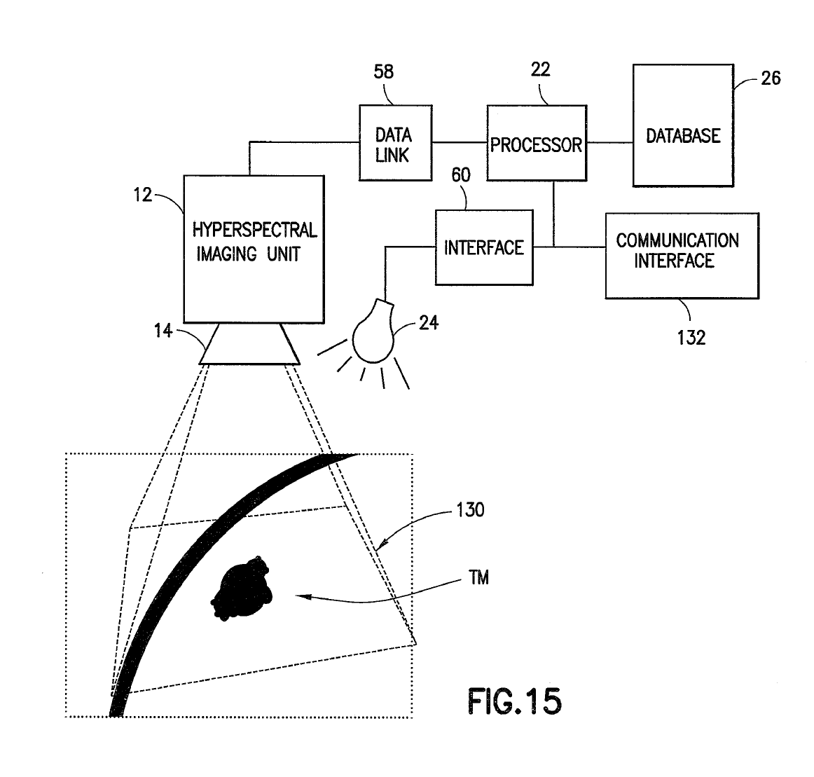

The present invention is directed to hyperspectral imaging systems, units, and methods, which, together with certain outputs, data structures, and displays, are illustrated in preferred and non-limiting embodiments in FIGS. 1-24.

In one preferred and non-limiting embodiment, the hyperspectral imaging systems, units, and methods described herein are applied to the field of biological (including organic) detection and medical diagnosis, and represent a non-contact, non-sampling, non-invasive, non-cooperative (and, potentially, remote) method of obtaining information that may include vital signs and other medically interesting measurements and features of biology, chemistry, physiology, and anatomy, such as a condition of biological material, e.g., normal or damaged tissue. The presently-invented hyperspectral imaging systems, units, and methods are particularly useful in connection with medical health monitoring and diagnosis, and represent an effective process for acquiring hyperspectral images that are medically useful and in a cost effective manner.

As discussed herein, and in certain preferred and non-limiting embodiments, provided are hyperspectral imaging systems, units, and methods having beneficial spatial and spectral resolution, reasonable exposure times and associated costs, and are easy to use and implement. The biological and/or medical information and data obtained or derived from the hyperspectral images of various visually-accessible bodily appearances can be useful in diagnosing physical conditions that may require medical intervention, or extend knowledge of what is “normal”. Accordingly, the presently-invented hyperspectral imaging systems, units, and method described herein provide a hyperspectral image acquisition system (e.g., a camera), a method of image analysis and feature identification (e.g., algorithms and processes), an assured connection between identified image features and normal versus pathological conditions (e.g., analysis and diagnosis), and an accumulation of a large number of such relationships (e.g., a database for use in comparison and data warehousing).

In certain preferred and non-limiting embodiments, the hyperspectral imaging systems, units, and methods described herein include certain sensor designs and methods of use, various methods of scanning or other approaches to acquire a sufficiently-resolved datacube, and high resolution spectra of defined areas within a conventional color image. As described herein, the hyperspectral imaging systems, units, and methods may be implemented in a variety of medical and biological applications, and provide processes for diagnostics of specific pathologies. Further, and as the data and information is gathered and analyzed, provided is a database of visual information correlated with specified medical and pathological properties.

Certain sensor array chips are presently available with varying properties, with CCD (Charge Coupled Device) and CMOS (Complementary Metal Oxide Conductor) representing the most common chips, and with (e.g. Bayer mask) or without individual or local area pixel filter masking (where monochrome may have better spatial resolution). The Foveon chip offers limited color resolution for each pixel enhancing spatial resolution but, while good for general color photography, has no great advantages for hyperspectral applications. Each chip technology offers advantages and these evolve relatively with improving designs. In summary, a CCD offers a larger energy capture fraction and serial readout with minimal local processing, whereas a CMOS has addressability and processing capability for each pixel, but with some loss of sensitivity.

An important consideration for any two-dimensional pixel array is based upon how many photoelectrons may be derived from each pixel during the exposure time. In particular, for a finite exposure, the available resolution may be dependently distributed by design across the spectral resolution, and the two dimensions of spatial resolution for each elementary scalar point in the three-dimensional datacube. The achieved signal-to-noise ratio (SNR) will typically differ from point to point as the square root of the number of photoelectrons released. Exposure is typically constrained by constancy of image properties, image position, available integration time, and available light. It is further noted that the polarization of light may carry potentially useful information. In one preferred and non-limiting embodiment, the hyperspectral imaging systems, units, and methods described herein may provide a datacube with edge resolutions that determine the ability to acquire and subsequently process medically relevant diagnostic information, from tissue health to potentially life-threatening warning signs not immediately apparent even to a trained eye.

In another preferred and non-limiting embodiment, the hyperspectral imaging systems, units, and methods of the present invention may use or implement an improved method and process for spatial resolution enhancement (as described in Patent Cooperation Treaty (PCT) Application Serial No. PCT/US2013/023711, entitled “Spatial Resolution Enhancement in Hyperspectral Imaging” (filed Jan. 30, 2013 under attorney reference number 6709-123945), which is incorporated by reference herein in its entirety). This process can be implemented to maximize and subsequently redistribute the finite amount of available image information into areas of optimized diagnostic effectiveness. As also described herein, provided are processes and methods for image analysis for optimizing medically-relevant feature extraction, with relation to specific pathological aspects of the observed scene. Accordingly, and in further preferred and non-limiting embodiments, provided are hyperspectral imaging systems, units, and methods that: (1) enhance the distribution of the acquired information within the datacube to maximize the medical diagnostic benefit; (2) determine or identify medically- or biologically-useful information and data based upon the hyperspectral image; (3) provide effective and implementable acquisition and analysis systems and units to obtain a specified datacube or datacubes with automatically and/or intelligently extractable features.

With respect to the described hyperspectral imaging systems, units, and methods, it should be noted that the resolution may be distributed in many different ways. In general, the number of available pixels in each of the two image dimensions (conventionally “x”, and “y”), is a simple linear scaling factor, since in any single commercial product almost all individual pixels are typically the same size (although they may vary in both shape and size between devices (square, rectangular, stripes, etc.)). The “filling factor” is also uniform across the chip but can vary between “x” and “y”, indicating a possible benefit of orientation for some scene properties. Spectral discrimination is more complex, offering many choices for optimization specific to an application. The free parameters for an equivalent array of spectrally resolving elements are overall wavelength sensitivity range, number of equivalent filters, peak wavelength, and transmission band profile of each individual filter. Filter overlap is already subsumed in the preceding four parameters. The values of these parameters are not constrained to be constant nor uniform across the spectral range, which may itself exceed the human visual wavelength sensitivity range, and may be optimized for any specific purpose, as is ubiquitous throughout the animal kingdom where many methods are found according to the requirement of the animal.

Possibilities for distributing the spectral resolution vary from a serially substituted array of individual filters, such as were exploited in the early days of color television, through optical dispersive devices, such as diffraction gratings with suitable optics, to filters for individual pixels, such as the Bayer mask, or more sophisticated devices based upon carefully designed and configured arrays of deposited Fabry-Perot stacks. As described in the above-mentioned PCT Application No. PCT/US2013/023711, processes and methods have been developed to provide an optimized spatial/spectral resolution compromise by distributing individually chosen Fabry-Perot filters across a base chip, either covering individual pixels or blocks of pixels in optimally-selected patterns, such a Walsh-Hadamard, or “magic square” patterns, of which there is almost an infinite number of possible variations.

In further preferred and non-limiting embodiments, provided are hyperspectral imaging systems, units, and methods that are useful in imaging and analyzing a variety of visually-accessible medical and/or biological conditions. For example, the hyperspectral imaging systems, units, and methods of the present invention can be used to observe a dermatological condition at high spectral resolution and/or outside the range of the human visual observer. This capability, however implemented, may be extended to other regions optically accessible with minimal intrusion, such as the ear, eye, nose, throat, gastrointestinal tract, genitals, anus, and acute and chronic wounds. Further, it is envisioned that certain preferred and non-limiting embodiments of the hyperspectral imaging systems, units, and methods can be used in connection with invasive camera systems, such as those used for surgery, internal visualization after trauma, laparoscopic investigations, and/or medical disease screenings.

In another preferred and non-limiting embodiment, the hyperspectral imaging system, unit, and method of the present invention is implemented by modifying a conventional color camera to split off part of the incoming image. In this embodiment, and as designated by a crosshair or other target identifier, a defined region of the observed scene presents light through a high resolution miniature spectrometer. Moving the camera across the scene shows a conventional visual image from a small defined region of which is simultaneously displayed a high-resolution spectrogram. Such extended capability may permit the diagnosis of pathological conditions not visually obvious. The ability to select specific regions on the basis of the usual color image may lead to greatly extended ability to determine the nature of skin conditions, wounds and their healing, chemical signatures of disease or from damage and other detrimental conditions whose detected presence may allow medical diagnosis and/or remediation.

In still further preferred and non-limiting embodiments, the hyperspectral imaging systems, units, and methods provide imaging at various spatial scales that is useful for examination and characterization with and without noticeable anomaly of various medically interesting properties: (1) accessible skin, including all aspects of dermatology (moles, seborrheic keratosis, actinic keratosis, basal cell carcinoma, squamous cell carcinoma, melanoma, other skin neoplasms, keloids, scars, wounds, telangiectasias, acne rosacea, cysts, psoriasis, vascular growths, hyperpigmentation, hypopigmentation, vitiligo, eczema, dermatitis, onychomycosis, tinea, hair changes, nail changes, etc), areas of discoloration or non-uniformity, eye, including iris, cornea, sclera and surrounding tissue, mechanical damage and bruising from injury and trauma, physical damage from burns, level of hydration and edema, progress of wound healing and scar formation, bilirubin levels, skin and tissue oxygen and blood perfusion, fungal growth and infestation conditions, response to allergy induction tests, and/or perfusion and viability of surgical flaps; (2) other externally accessible locations, including auditory canal and tympanic membrane, eye, including humors, internal structures and retina, mouth, tongue, tooth, gum, throat and mucus membranes, nasal cavities and membranes, other mucosal surfaces including genitals and anus, gastrointestinal tract and organs thus accessible, and/or physiological states and facial dynamics; (3) less accessible and partially invasive locations, including vagina, uterus, urethra, rectum, colon, liver, kidney, pancreas, intestines, lung, fetus, bladder, cervix, uterus, and/or testicles; (4) invasive imaging typically involving surgical incisions to allow access, including liver, kidney, pancreas, intestines, testicles, and/or any and all other organs and structures optically accessible to devices modified from one or more of a cystoscope, laparoscope, colonoscopic camera, sigmoidoscope, cardiac, and/or endothelial vascular catheter, surgical camera, microscope, or other imaging or color sensing device applicable to internal and external inspection (e.g., fetus evaluation), surgical procedures, and other such bodily invasions; (5) specimen appearance, including color and content of urine, blood, sputum, tears, mucous, semen, amniotic fluid, and all other bodily fluids; (6) test analysis, including characterization of test strips, liquids, semi-solids, agar, etc., and/or chromatic and electrophoretic indications; (7) microbiology assays, including characterization of bacterial growth and speciation, and/or atypical mycobacterium growth; and/or (8) biochemical assays, including ELISA reports and/or enzyme activity assays.

Following is a description of certain technical characteristics and properties that are used and/or implemented in connection with certain preferred and non-limiting embodiments of the hyperspectral imaging systems, units, and methods of the present invention.

The fluence (the product of irradiance, or radiant flux, and time) incident upon a pixel in the image plane stimulates a detector to produce an electrical signal, which may be characterized generally as a number of emitted photoelectrons Nₚ(x,y), whose implicit shot noise is the square root of this number. This can be calculated as:

$${N}_{P} ( x , y ) = {\int }_{0}^{T} {\int }_{0}^{Ω} {\int }_{x - \frac{δ x}{2}}^{x + \frac{δ x}{2}} {\int }_{y - \frac{δ y}{2}}^{y - \frac{δ y}{2}} {\int }_{λ - \frac{δλ}{2}}^{λ + \frac{δλ}{2}} R ( x , y , λ , θ ) \text{?} F ( x , y , λ , θ ) \text{?} S ( x , y , λ , θ ) d λ d y d x d θ d t$$

$$\text{?} \text{indicates text missing or illegible when filed}$$

where R(x,y,λ,θ) is the spectral radiance of the scene as imaged on a pixel of dimensions δx,δy at a location x, y in the image plane of the detector, at an elementary peak wavelength λ with an approximated average bandwidth of δλ, at an angle θ to the principle ray of an assumed axi-symmetric optical system without sensitivity to polarization. F(x,y,λ,θ) is the transmission coefficient of the equivalent filter preceding the element x,y at an elementary peak wavelength λ with an approximated average bandwidth of δλ, and may have a residual angular dependence upon θ (vignetting). The angular dependence is assumed to be circularly symmetric, but this need not be so as this filter transmission term may be adjusted in λ and θ to accommodate any limitations of the optical system that may vary over the observation aperture or across the detector array field. The final significant term, S(x,y,λ,θ) characterizes the properties of the detecting element, such as quantum efficiency and sensitivity, which will usually have a predictable dependence on wavelength over the specified interval, and if sufficiently uniform any residual dependence upon x,y, may be calibrated for correction or compensation. Angular dependence may however remain, particularly if the optical system is “fast”, i.e., with a large cone angle incident upon the filter and/or detector, to establish the large “Lagrange Invariant”—so desirable where the amount of light is at such a premium. The term “filter” as used herein indicates spectrally resolved bands but, of course, any dispersive element, such as a reflective ruled or holographic grating, is equally accommodated by F(x,y,λ,θ).

If polarization is significant then the integral above may be modified to include the additional term Cos²(ξ_{B}(x,y,λ,θ)−ξᵢ(x,y,λ,θ)) where ξ_{w}(x,y,λ,θ) is the polarization orientation of the scene emission, and ξᵢ(x,y,λ,θ) is the polarization orientation of sensitivity in the detector and/or filter element combination at x,y. In almost all situations of acceptable optical design, this term can be reduced to near unity, unless the deliberate choice is made to make the system polarization sensitive. Many animals exploit this for various advantages. In certain optical systems, the relationship of detector irradiance to scene radiance approximates to I(x,y,θ)=ΩR(x,y,θ), representing the conservation of étendue, a slight modification of the Lagrange invariant, which is mostly conserved though an optical system where object and image are immersed in a medium of the same refractive index, and there is no vignetting from any cause. It may be important to retain the additional conceptual sophistication because the transmission of typical narrow band filters is not usually independent of angle.

For hyperspectral imaging, the interpretation of the above equation and the associated ability to calculate signal and signal-to-noise ratio (SNR) is straightforward because all terms are linear and invertible. For example, the forward transform may be calibrated, and its inverse used to find the scene properties directly in terms of the measurable photon flux, or the equivalent integrated charge. If the scene is only passively and linearly reflective then its radiance is determined by the intensity of the illumination and its specific spectral reflecting or scattering properties. Since many materials of interest in hyperspectral imaging are Lambertian or quasi-isotropic scatterers, the spectral signature of the examined material may be derived by dividing the measured spectrum by the illumination spectrum, being careful that the associated eigenvectors are well clear of the noise floor—that is, that one does not inadvertently divide by too small of a number in the inversion process. Should the specimen exhibit fluorescence, then this is slightly more complex; but, even the spectral spread and non-linearity of the emitted wavelength spectrum need not impair the ability to extract any required properties. Thus, for a chosen detector array and material, provided are two independent controls to determine the hyperspectral nature of a specimen. The illumination can be varied, as well as the filter arrangements, which may be implemented in a variety of manners, each with its own advantages and disadvantages.

With respect to measurement, the illumination properties L(x,y,λ,θ) are used in connection with the measurement of R(x,y,λ,θ) and since only the specimen properties are required, referred to as T(x,y,λ), without angular dependence, it is considered that the results of the original measurement is divided by this independently determined quantity. This is a property of calibration, which will not only compensate for a number of assumptions that are not perfectly valid, but also allow the extraction of the required specimen properties by division on an equivalent pixel-by-pixel or integrated local area of spatial resolution basis. Although certain variations still exist based upon a variety of reasons, including, but not limited to design approximations, temporally varying image, movement blurring, dynamic intensity range, optical system properties, such as focus over the field, aberrations, and the like.

With respect to signal strength, and in connection with the hyperspectral imaging systems, units, and methods of the present invention (involving biological and/or medical applications), certain simplifications and extensions can be implemented. First, considering a single pixel reduces the above equation to:

$${N}_{P} = {\int }_{0}^{T} {\int }_{0}^{Ω} {\int }_{λ - \frac{6 λ}{2}}^{λ + \frac{6 λ}{2}} \frac{T ( λ ) , F ( λ , θ ) , S ( λ , θ , )}{L ( λ , θ )} d λ d θ d t .$$

For a fixed instrument, a stationary standard specimen can be viewed, e.g., a “Spectralon” and assuming that the system is linear enough to perform the proper and constant integral over a solid angle, albeit not ideally uniform, yields:

$${N}_{P} = Ω {\int }_{0}^{T} {\int }_{λ - \frac{δλ}{2}}^{λ + \frac{δλ}{2}} \frac{T ( λ ) , F ( λ ) , S ( λ )}{L ( λ )} d λ d t .$$

While this represents the beginning of the ability to calculate signal, two further conditions are necessary for the above approximation to be acceptable, namely: (1) the value of collection solid angle transformed into the image space must not exceed the value over which the spectral properties of the equivalent filter bandwidth are quantified to be acceptable—this is equivalent to slit width in grating spectrometers; and (2) the “filters” if implemented as Fabry-Perot interferometers must have sufficient free spectral range to accommodate without aliasing the band of wavelengths required. If a larger wavelength range is required, then the same filters may be used at different aliasing orders with suitable blocking filters, which may typically be absorptive.

To completely cover a certain free spectral range with a given number of Fabry-Perot filters, each filter width may be adjusted by the finesse

$$\frac{4 R}{( 1 - R \text{?}}$$

$$\text{?} \text{indicates text missing or illegible when filed}$$

of the device, where R is the surface reflectance of the bounding layers of each stack. These stacks need not be the same width, nor have a similarly shaped transmission envelope, nor be separated by a constant wavelength interval, but may be tailored to a specific application, much as evolution has achieved such optimizations throughout the animal kingdom. In one preferred and non-limiting embodiment, the process for physically distributing the pattern of independent filters is described in the above-mentioned PCT Application No. PCT/US2013/023711, although other approaches are available, such as the use of a linear array of linear arrays, thereby approximately simulating the properties of a dispersive spectrometer.

Given the above, and based upon the number of parameters, it may be difficult to obtain the datacube simultaneously, but the parameterization of the axial resolutions remains a subject for optimization. A complete datacube may contain a single scalar value of fluence Nₚ (the product of signal and time, expressed as number of emitted photoelectrons) at each cell of dimensions δx,δy,δλ (which need neither be uniform nor uniformly distributed) over the three orthogonal fixed axes of x,y,λ. Nₚ increases with exposure time, and reciprocity failure may be prevented with proper configuration and use of either CCD or CMOS sensing array devices. If, therefore, a given minimum value and/or range of values of Nₚ is acceptable, the acquisition methodology can be distributed among the four remaining free parameters x,y,λ,t.

In one preferred and non-limiting embodiment, and for a dispersive spectrum with a linear detector, all values of x and λ are simultaneously available, y=1, and the image field is swept-scanned at a rate sufficient to get enough signal for each dwell time of t, for a total time of T=yt. An existing method may be referred to as the “push-broom” method. If the one-dimensional linear array is replaced by a two dimensional array, the push-broom method will take only one scan time instead of the many required in the first example. With this approach, all values of y are measured (albeit at different values of λ) simultaneously for a total comparable acquisition time of T=t, yielding a substantial improvement. It should be noted that with either linear filter array, one should wait until the end of the scan before accessing a complete datacube; but, that intermediate information is available during the acquisition in rather different ways. For “push-broom” systems with a line image dispersed on to a two-dimensional array detector, an image of the whole scene is obtained even before scanning, but with each line segment viewed through a different filter. Morphological features that have components at all wavelengths are thus immediately identifiable, but to see the whole image at all wavelengths the line scan must cover the image field, requiring redundant coverage of twice the single image area to be examined over the full wavelength range.

Changing the filter pattern from lines to an optimized array is capable of realizing immediately the low resolution aspects of the whole image at all wavelengths, with a now-lesser scan range building the datacube differently (but potentially more usefully), in that hyperspectral data are acquired with steadily increasing spatial resolution—somewhat analogously to the quad enhancement of progressive JPEG encoding. The fundamental difference is that the acquisition of the same full datacube proceeds along different paths through the four-dimensional space, permitting different features to be identified earlier in the scan. This may have advantages for moving or changing images, or where an immediate rapid low spatial resolution view may be more useful than a more complete datacube that requires a more stable image at the highest possible resolution, but over the full observation time.

One preferred and non-limiting technique to avoid the need to scan twice the area with a linear pattern is to distribute the linear filters radially and rotate the image once—a saving of a factor of two on comparable image acquisition time. It should be further noted that for the same light level and transmission bandwidth, the region near the point of rotation has higher resolution, substantially like the fovea centralis in humans and some other animals. A miniature asymmetric mirror Dove prism would readily accomplish this rotational scanning, but incurs the disadvantage of moving parts. Both of these two previous modalities assume a uniform illumination. If the illumination is changed between integration frames, there are two additional alternatives: (1) use a monochromatic sensor and cycle though as many illuminants as spectral bands in the same time T=t; or (2) use a standard Bayer mask CMOS, and get the same data with about T=t/4 with only marginal reduction of the spatial resolution. However, it should be considered that there should be as many different narrow band illuminating colors as there are hyperspectral bands, and a suitable distribution of source wavelengths and power may not be readily available at a reasonable cost.

In certain preferred and non-limiting embodiments, the hyperspectral imaging systems, units, and methods allow for, and readily implement, calibration before measurements that are to be accumulated into the standardized database. Such traceability of these comparative measurements lends itself to acceptability within the medical community. Accordingly, also provided may be an end-to-end calibration technique.

With reference to the schematic diagram of FIG. 1, and in one preferred and non-limiting embodiment, provided is a hyperspectral imaging system 10 that includes one or more hyperspectral imaging units 12. The hyperspectral imaging unit 12 includes one or more lenses 14 configured or operable to direct light L scattered by, reflected from, and/or transmitted through a target medium TM (and/or region of interest 130 or portion thereof) to one or more hyperspectral filter arrangements 16. As used herein, the hyperspectral filter arrangement 16 refers to one or more hyperspectral filters, one or more hyperspectral filter arrays (e.g., an array of narrow-band filters), one or more sets of hyperspectral filters, one or more filter elements, one or more dispersive elements, any element configured for the high resolution separation of colors, and the like. The hyperspectral filter arrangement 16 is configured or operable to separate the received light into multiple discrete spectral bands. The hyperspectral imaging unit 12 also includes one or more imaging sensors 18, which are configured or operable to: (a) receive the discrete spectral bands from the hyperspectral filter arrangement 16; (b) detect light by or for multiple pixels for each of the spectral bands; and (c) generate electrical signals based on at least a portion of the light. Further, the hyperspectral imaging unit 12 of this preferred and non-limiting embodiment includes one or more image processors 20 that are in communication with the imaging sensor 18 and configured or operable to generate hyperspectral image data that are associated with the target medium TM (and/or region of interest or portion thereof). Still further, the hyperspectral imaging system 10 includes one or more processors 22 (whether integrated with or remote from the hyperspectral imaging unit 12) configured or operable to determine biological data, such as biological or organic condition information or data, based at least partially on at least a portion of the hyperspectral image data. It is recognized that any of the components of the system 10 and unit 12 described herein can be rearranged and/or integrated to obtain the desired hyperspectral data and/or biological data and information.

With continued reference to the preferred and non-limiting embodiment of FIG. 1, the hyperspectral imaging system 10, and in particular, the hyperspectral imaging unit 12, includes one or more light sources 24 that are configured or operable to direct light L towards or through at least a portion of the target medium TM (or, as discussed in greater detail hereinafter, a region of interest 130 or portion thereof). This light source 24 may be in a variety of preferable forms, including, but not limited to, a light emitting diode, a laser, a colored light source, a configurable light source, and the like. Further, the light source 24 may be in the form of ambient lighting (where it is not a separate physical component of the unit 12), such as in the case where the target medium TM is translucent. Still further, this light source 24 may be in the form of a unit configured to produce or provide light at any wavelength, such as at any wavelength between the ultraviolet and the infrared.

In another preferred and non-limiting embodiment, the hyperspectral image data are at least partially in the form of or include a hyperspectral datacube that is made up of multiple images, where each image is in the form of or represents a discrete spectral band. For example, the hyperspectral image datacube may include an X-axis, a Y-axis, and a wavelength axis. In addition, it is understood that the hyperspectral data may include a datacube that is not limited to separate slices in the form of cross-sections in the wavelength planes. In particular, and as discussed and used herein, the “datacube” may be built from slices in any of the three axes, such as by accumulating arrays of pixels distributed throughout the cube, each of which is displaced at random in the X and/or Y axes (but not according to wavelength)—because the filter mask is constant. Still further, the datacube may be time-resolved, such that the hyperspectral data include a four-dimensional data acquisition for the target medium TM or region of interest 130. Accordingly, the hyperspectral data generated, processed, and/or determined by or within the system 10 and unit 12 of the present invention may include any type of useful visual, spectral, hyperspectral, video, and/or image information. As also seen in the preferred and non-limiting embodiment of FIG. 1, the hyperspectral imaging system 10 may include a hyperspectral database 26, which is populated with existing hyperspectral image data and/or associated biological data or medical information. This database 26 may be part of or accessible by the processor 22. In addition, the biological data may be determined by comparing at least a portion thereof (and/or any of the hyperspectral image data (as generated by the hyperspectral imaging unit 12 and/or the processor 22)) with the existing hyperspectral image data in the database 26. Based upon the existing association between this existing hyperspectral image data and associated biological data, such comparison can result in providing a diagnosis or other medical and/or biological determination about the biological condition of the target medium TM or region of interest 130, typically of a user or patient.

In another preferred and non-limiting embodiment, and as illustrated in FIG. 1, the hyperspectral imaging unit 12 may also include a communication interface 28 that is configured or operable to communicate at least a portion of the hyperspectral image data to the processor 22, such as a remote processor. This communication interface 28 may also be in communication with or otherwise integrated or associated with the image processor 20. Of course, this communication interface (or any of the components discussed above in the hyperspectral imaging unit 12) may be a separate component.

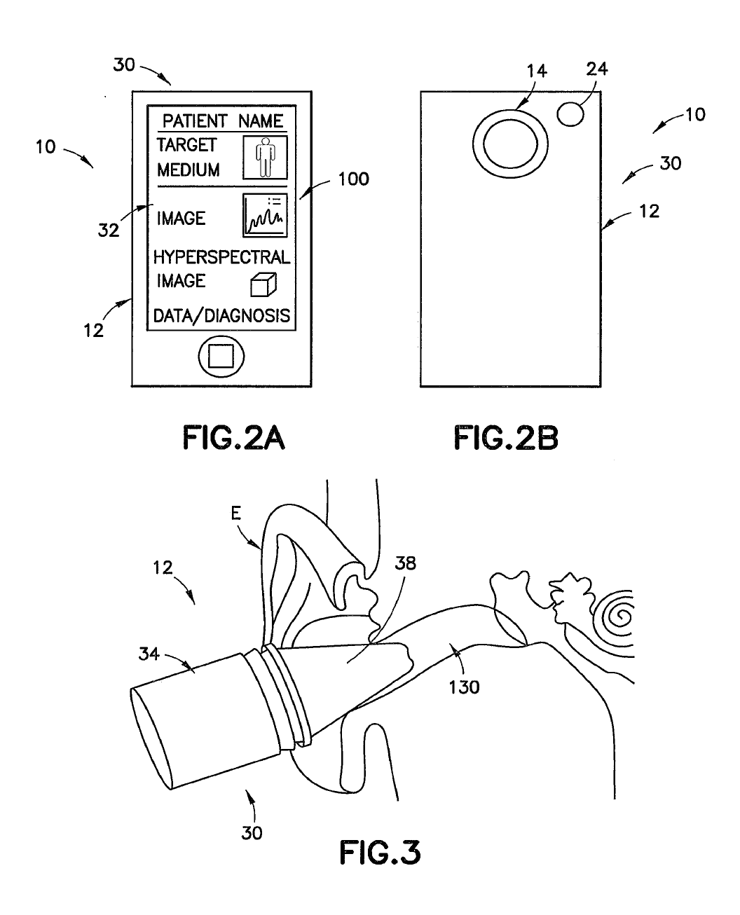

Another preferred and non-limiting embodiment of a hyperspectral imaging system 10 according to the present invention is illustrated in FIGS. 2(a) and 2(b). As is seen and in this embodiment, the hyperspectral imaging unit 12 comprises, is part of, or is integral with a portable device 30, such as a handheld device or the like. In addition, it is further envisioned that the processor 22 may also be part of or integrated with this portable device 30. Of course, in other embodiments, the portable device 30 is used to collect and determine the hyperspectral image data and provide this data to a remote or separate device having the above-discussed processor 22. Such communication may occur in either a hard-wired (e.g., a docking device) or wireless format and infrastructure. Accordingly, this portable device 30 may be a cellular telephone, a smart phone, a laptop computer, a pad computer, a handheld computer, a personal digital assistance, a portable electronic device, and the like.

With continued reference to the preferred and non-limiting embodiment of FIG. 2, specifically FIG. 2(a), the portable device 30 may be in the form of a smartphone having a display device 32, e.g., a screen, configured or programmed to display information and data. In this particular application, any of the above-discussed information and data can be displayed to a user of the portable device 30. In addition, this display device 32 may be interactive with or otherwise allow the user to provide input to and receive data from the display device 32 and/or the portable device 30. Accordingly, any of the raw, pre-processed, or processed data described above, e.g., light information, electrical signals, hyperspectral image data, biological data, user data, patient data, configuration data, control data, and the like can be formatted and displayed on the display device 32. Further, the display device 32 may also function as the input mechanism to allow the user and/or patient to control any component of the portable device 30, the hyperspectral imaging unit 12, the hyperspectral imaging system 10, or any combination thereof. In another preferred and non-limiting embodiment, the portable device 30 is either loaded with or otherwise in communication with the above-discussed database 26, such that the determination of the biological data or any resultant or generated data from the system 10 can be generated and displayed on the display device 32.

With respect to FIG. 2(b), and in this preferred and non-limiting embodiment, the hyperspectral imaging unit 12 is integrated with or included with the portable device 30, where the lens 14 or lens system is on the rear surface of the portable device 30. In addition, this portable device 30 includes a light source 24 or components thereof, such as a flash device, as is known in the art. It is further envisioned that the typical camera included with known portable devices 30, e.g., a smartphone, can be used, modified, or replaced with an appropriate hyperspectral imaging unit 12, or any component thereof. Accordingly, the preferred and non-limiting embodiment of FIG. 2 provides an easy-to-use handheld hyperspectral imaging unit 12 and hyperspectral imaging system 10 for gathering, processing, generating and/or displaying appropriate data.

In another preferred and non-limiting embodiment, and as illustrated in FIG. 3, the hyperspectral imaging system 10 and hyperspectral imaging unit 12 are used to determine biological data including biological condition information relating to a person's ear E, such as a patient's outer ear, middle ear, and the like. In addition, this biological condition information may be related to the nasal cavity, the patient's throat, or any other bodily surface or region that is optically accessible. In this embodiment, the hyperspectral imaging unit 12 includes a housing 34 with an end 36 that is shaped or configured for at least partial insertion in a patient's ear E, outer ear, middle ear, nasal cavity, throat, and the like. As is seen in FIG. 3, the end 36 of the housing 34 includes a conical tip 38 that is shaped for partial insertion in the patient's ear E for gathering hyperspectral image data about the ear E, which are then used to generate biological data, such as biological condition information, related to the ear E. Of course, this conical tip 38 can be modified in shape or design for insertion in other parts of the body, including, but not limited to, the patient's nasal cavity or throat.

Accordingly, this preferred and non-limiting embodiment allows a user to take hyperspectral images of the middle ear, as a normal otoscope is used, such that the biological information and/or biological condition information allows for the detection of ear infections, or indication of conditions not normally accessible to conventional visual observation. Of course, as discussed above, this hyperspectral imaging unit 12 may also be used in connection with the nasal cavities and/or throat of a human or animal. In addition, the housing 34 may include an elongated portion, handle, or other area, that is easily graspable by the user. Accordingly, in this preferred and non-limiting embodiment, the hyperspectral imaging unit 12 is in the form of an electronic otoscope, which generates visible, digital images of the auricular and/or nasal cavities, with superior per-pixel spectral resolution compared to existing otoscopic devices. This superior per-pixel spectral resolution will manifest as an enhanced ability to detect the spectral characteristics at each locus of the examined auricular and/or nasal cavity, and this enhanced ability to detect the spectral characteristics at each locus of the examined auricular and/or nasal cavity may result in an improved ability to detect and diagnose infections and/or other maladies of the outer and middle ear, or of the upper respiratory system, such as based upon a comparison of hyperspectral data and/or biological data in the database 26.

With continued reference to the preferred and non-limiting embodiment of FIG. 3, the hyperspectral imaging unit 12 facilitates the spectroscopic scanning of the auricular and/or nasal cavities at wavelengths that may be outside the visible, thereby generating a visual, digital image that indicates the spectroscopic scattering and/or reflectance properties of all loci of the cavities within and beyond the visible spectrum. As used in this preferred and non-limiting embodiment, but applicable to other embodiments, the hyperspectral imaging unit 12 includes a process where each extra-visible wavelength is assigned and may be displayed as a visible or “false” color or pseudo-color, based upon the use of the hyperspectral imaging sensor 18 and system 10 instead of a conventional monochrome or RGB image sensor. In addition, the hyperspectral imaging unit 12 may be configured to store and/or transmit the hyperspectral image data “as-is,” as opposed to generating a false-color image, and allowing feature detection algorithms to be applied on the raw, pre-processed, processed, and/or post-processed hyperspectral image data.

As discussed, the hyperspectral imaging unit 12 of FIG. 3 allows for the examination of the patient's ear E, such as the outer and middle ear, but may also be used to examine the nasal cavity, upper throat, or any other bodily surface or region that is optically accessible. Further, the hyperspectral imaging unit 12 may include the appropriate components, such as color-neutral light detection elements, for achieving the appropriate resolution. Hyperspectral imaging may also occur without any dispersion, where, on each pixel element of the underlying monochromatic imaging sensor 18, only a small range of the spectrum is filtered through on each pixel. In another preferred and non-limiting embodiment, the hyperspectral imaging unit 12 employs a stepwise-wedged filter arrangement 16, which may be less expensive to produce, while still enabling superior spectral or hyperspectral resolution to that of conventional imaging systems. This filter arrangement 16 may be a per-pixel narrowband filter arrangement, a Fabry-Perot filter arrangement, and the like. In addition, the use of hyperspectral imaging, as opposed to digital color photography, provides superior spectral resolution by dividing the visible spectrum into a far larger number of spectral bands, as well as maintaining the separation of these spectral bands, thereby producing full hyperspectral (e.g., spectral or reflectance data) at each pixel. Further, while digital color photography technology separates the sample data into three spectral bands, i.e., red, green, and blue (RGB), the hyperspectral imaging system 10 according to the present invention divides the sample spectrum into tens, hundreds, or even thousands of bands, typically with a ten nanometer or less bandwidth. This higher resolution spectral data and information can be added together within each pixel, as occurs with RGB-based systems, but with greater color resolution, to produce a more perfectly resolved “overall” color at each pixel. Alternatively, the full spectral data can be observed at each pixel, resulting in a maximal picture of surface absorbtance, reflectance, and/or scattering for the examined target medium TM or region of interest 130; in this embodiment, the patient's outer and middle ear E, nasal cavity, upper throat, or any other bodily surface or region that is optically accessible.

Still further, and with reference to the present embodiment, or other embodiments (as discussed hereinafter), the hyperspectral imaging system 10, unit 12, and methods discussed herein provide the ability to scan a broad spectral range. Depending in part on the nature of the incident light source 24, the tuning of the hyperspectral imaging unit 12, and/or the filter arrangement 16 used, the hyperspectral imaging sensor 18 can scan sample reflectance data in spectral regions including, potentially, the ultraviolet, the visible, near-infrared, medium-infrared, far-infrared, and beyond. Further, as discussed above, the extra-visible spectral data could be added at each pixel to produce a color map, where extra-visible wavelengths are assigned visible colors to produce a visible image of the extra-visible hyperspectral data. Alternatively, full spectral hyperspectral data could be recorded at each pixel.

With continued reference to the preferred and non-limiting embodiment of FIG. 3, but with equal application to other embodiments discussed above and hereinafter, the hyperspectral imaging systems 10, units 12, and methods provide additional advantages. First, healthy and unhealthy tissues can have minor differences in their composition and identity of proteins, nucleic acids, and a multitude of other various chemicals and bio-molecules they contain. Such compositional variations can be undetectable to standard visible examination using traditional equipment, such as a “lens and eye” otoscope, or through a standard digital imaging otoscope. Such compositional variations can, however, be expressed as slight shifts in the peaks and troughs within a spectral curve. This can enable a distinction between healthy and unhealthy tissues that is not possible with conventional visual inspection. In addition, the possibility of scanning wavelength ranges outside the visible further enhances the diagnostic power of the presently-described hyperspectral imaging system 10, unit 12, and method.

As illustrated in FIG. 3, the conical tip 38 of the housing 34 is inserted into the human ear E and protrudes into the middle ear canal. It is envisioned that the components discussed above in connection with FIG. 1 and the hyperspectral imaging unit 12 described in connection with FIG. 1, may also be used, included with, or integrated into the housing 34 of the preferred and non-limiting embodiment of FIG. 3. Also, in one preferred and non-limiting embodiment, the communication interface 28 can be wireless (e.g., Bluetooth, ZigBee, IEEE 802.11), or wired (e.g., USB or accessory cable).

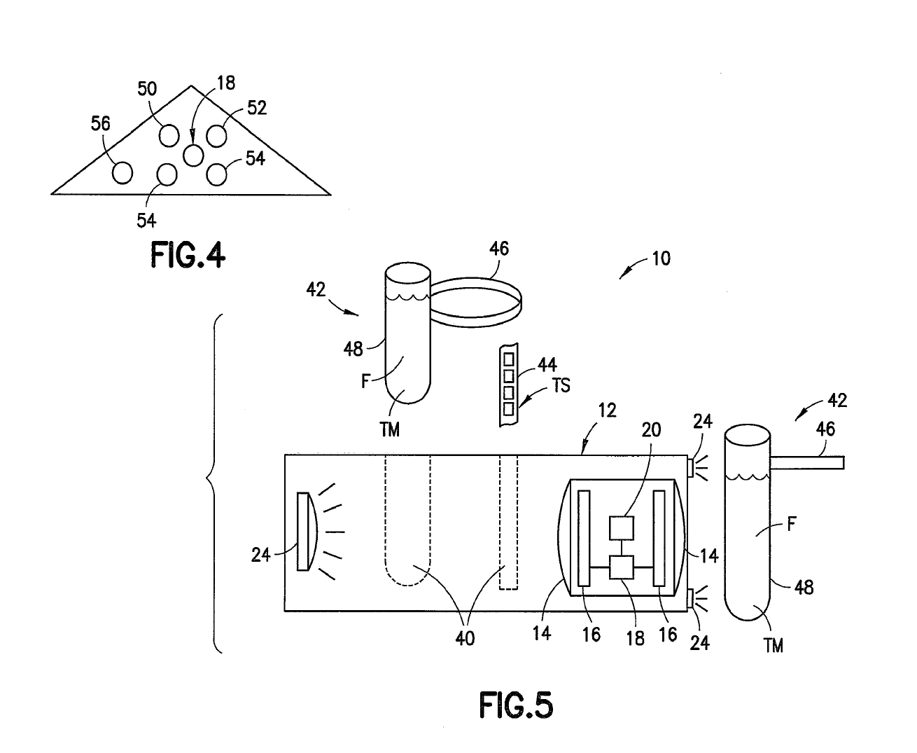

In further preferred and non-limiting embodiments, and with reference to FIGS. 4-6, the biological data include biological condition information relating to a fluid, bodily fluid, blood, urine, saliva, sweat, semen, mucus, and the like. In the preferred and non-limiting embodiment of FIG. 5, the hyperspectral imaging unit 12 may include an insertion portion 40 configured or adapted to at least partially receive a collector 42, a test strip 44, a container 48, and the like. As is illustrated, the collector 42 (or container 48) may include or hold the fluid F, including any of the above-described fluids, slurries, partially liquid substances, mixtures, and the like. Accordingly, the collector 42 or container 48 may be inserted in or otherwise placed in the insertion portion 40 of the hyperspectral imaging unit 12, where the hyperspectral image data are generated using the components described above, which will be included with or otherwise housed by or in the unit 12. As also illustrated in FIG. 5, the collector 42 or container 48 may include a handle 46 for use in inserting the collector 42 at least partially into the insertion portion 40. In addition, this handle 46 may be arranged on or near the hyperspectral imaging unit 12, such that the hyperspectral image data can be collected without the use of the insertion portion 40.

As is known, standard urinalysis is accomplished through a combination of direct urine visual observation, urine dipstick analysis, a microscopic urinalysis. There are numerous methods of urine collection, which include random collection (taken at any time of day with no precautions), clean catch (mid-stream after cleaning of the urethral meatus), and catheterization (Foley or suprapubic). According to one preferred and non-limiting embodiment, the system 10 and unit 12 of FIGS. 4-6 is be used for evaluating urine collected through any of these methods. The first component of urinalysis is macroscopic assessment, where, through the naked eye, the color and turbidity (i.e., cloudiness) of urine is determined. Normally, fresh urine is pale to dark yellow in color, and is otherwise clear. The second component of the urinalysis is urine dipstick chemical analysis. Multiple urine parameters are evaluated via dipstick analysis, including urine pH (normal range of 4.5-8.0), urine specific gravity (normal range of 1.002-1.035), urine protein, glucose, ketones, nitrite, and leukocyte esterace. The third component of the urinalysis is microscopic assessment, where a sample of well-mixed urine is centrifuged to produce a cellular sediment, which can be re-suspended on a glass slide for visualization. The urine is then evaluated for the presence of red blood cells, white blood cells, epithelia cells, urine casts, crystals, bacteria, and yeast.

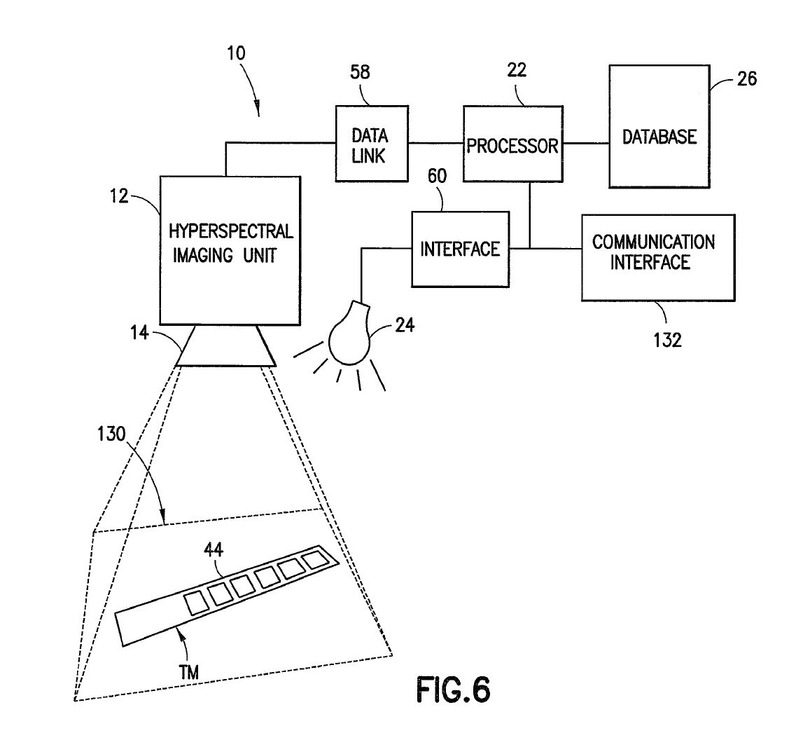

With reference to FIGS. 5 and 6, the test strip 44 can be used either as inserted into the insertion portion 40 of the unit 12, or otherwise placed at or near the unit 12 to collect the hyperspectral image data. This test strip 44 is configured to contact the fluid F, and the test strip 44 is impregnated or coated with at least one chemical that is capable of reacting with the fluid F. Also, as discussed above, and as illustrated in FIG. 5, the collector 42 may be in the form of a container 48, which can be positioned with respect to the lens 14 and/or light source 24 of the hyperspectral imaging unit 12, or alternatively, the hyperspectral imaging unit 12 can be positioned with respect to the container 48.

In one preferred and non-limiting embodiment, the fluid F is in the form of urine, where high levels of protein, hemoglobin, and/or creatinine in a urine sample may be indicative of a disease or other biological condition. The levels of these chemicals and others, can be determined with well-known and simple analytical chemistry methods, such as spectrophotometry. Accordingly, the hyperspectral imaging system 10 and unit 12 according to this preferred and non-limiting embodiment is used for analyzing urine, and the system 10 includes other urine collection devices, such as the collector 42, container 48, and/or test strip 44. In one preferred and non-limiting embodiment, the hyperspectral imaging unit 12 is used to measure the amount of protein in a urine sample, and the unit 12 includes an imaging sensor 18 and/or image processor 20 configured to quantify light levels in the ultraviolet through the infrared bands. Accordingly, the components of this unit 12 may include any of the above-described lenses 14, filter arrangements 16, fibers, gratings, minors, image sensors 18, image processors 20, and the like. Accordingly, this system 10 may be used to diagnose a range of diseases by measuring the absorbtance, reflectance, scattering, and/or fluorescence of protein and hemoglobin species in the urine, which may facilitate the diagnosis of a kidney disease, kidney trauma, preeclampsia, and the like.

As illustrated in one preferred and non-limiting embodiment in FIG. 4, the hyperspectral imaging sensor 18 and/or lens 14 is essentially positioned with respect to multiple different light sources 24, including, but not limited to, a red light emitting diode 50, a white light emitting diode 52, one or more ultraviolet light emitting diodes 54, and a laser 56 (operating or providing light at any wavelength, such as a wavelength between the ultraviolet and the infrared). The use of these different light sources 24 allow for the appropriate quantification of light levels in a variety of bands.

As discussed, the hyperspectral imaging system 10 and unit 12 can be effectively used in a variety of applications, such as, and with respect to the preferred and non-limiting embodiments of FIGS. 4-6, fluid analysis. As also discussed, the system 10 and the unit 12 (and associated methods) can be used to detect disease indicators or other biological conditions and information in urine. As is known, proteins are chains of amino acids, and one of the main proteins that is often found in the urine of unhealthy or gravely ill people is albumin. In particular, they are found in high levels in the urine of people with certain medical conditions, including, but not limited to, preeclampsia, chronic kidney disease, and acute kidney failure. Urine collected from healthy individuals over a 24-hour period can contain as much as 35 milligrams of the protein albumin. By comparison, urine collected from a person with chronic kidney disease may be four times higher. Similarly, patients who have more than 30 milligrams of protein per gram of creatinine in their urine may have renal problems or preeclampsia. A ratio above 30 milligrams of protein per gram of creatinine is a definite sign of distress.

By using the above-described system 10 and unit 12, such as the system 10 and unit 12 of FIGS. 4-6, the user can collect a urine sample into a collector 42 or container 48. This urine sample can then be analyzed without removing it from the collector 42 or container 48, such as by placing the collector 42 or container 48 in the insertion portion 40 or otherwise in a specific position relative to the unit 12 (i.e., the lens 14 and/or light source 24 of the unit 12). Once the collector 42, test strip 44, and/or container 48 is appropriately positioned, a measurement can be made and the data recorded. It is envisioned that any one or more of the components of the hyperspectral imaging system 10 and hyperspectral imaging unit 12 may be part of a larger medical diagnostic device or system. In addition, the light source 24 can be configured as discussed above in such a way that light will scatter toward the lens 14, and this light source 24 may be a light emitting diode, an incandescent bulb, a laser, or some chemical element. In addition, the system 10 and unit 12 may include multiple light sources, as set forth as, for example, provided in FIG. 4. Further, the unit 12 of this preferred and non-limiting embodiment can be configured or adapted to measure reflected light, scattered ultraviolet light, scattered infrared light, light absorbtance, light reflectance, light scattering, fluorescence, and/or Raman emission. While, in one embodiment, the standard configuration for this unit 12 is in “reflectance mode,” in which three or more light emitting diodes and a camera sensor are mounted on the same circuit hoard, any similar arrangement can be used. In this embodiment, the circuit board faces toward the sample and projects light into the sample, and the amount of light that is reflected back towards the image sensor 18 at a particular wavelength is indicative of the analyte level.

Still further, the system 10 and unit 12 as described and set forth in FIGS. 4-6 is compatible with and can be used with a wide variety of standard urine specimen containers. To make a measurement through the transparent plastic wall of a urine collector 42 or container 48, the plastic should have a high level of transparency over all spectral ranges of interest. Accordingly, the collector 42 and container 48 may be a rectangular polystyrene cuvette, or a ladle with a rectangular polystyrene cup. In addition, the collector 42 and container 48 described above may be additionally beneficial in that there is minimal sample contact by the user, e.g., once the sample is collected into the collector 42 or container 48, it can be analyzed without being transferred into another vessel.

As described above, and with reference to the preferred and non-limiting embodiment of FIGS. 4 and 5, provided is a compact optical instrument that can be used to measure the concentrations of proteins and blood in urine. As discussed, when the unit 12 shines light onto a urine sample, some of that light will reach the imaging sensor 18, and the amount of 260 nanometer ultraviolet light that reaches the imaging sensor 18 (and/or lens 14) is indicative of the protein concentration of the sample. In operation, and to make a measurement, the user first collects a urine sample of between 10 and 250 milliliters into the collector 42 or container 48, which is manufactured from an optically-transparent plastic, such as polystyrene, polycarbonate, polymethyl methacrylate, or 4-methyl pentene. The user then holds or places the sensor with respect to the collector 42 or container 48 and triggers the measurement to occur. The unit 12 then illuminates the sample with a variety of light sources 24, and records the amount of scattered, reflected, transmitted, or fluoresced light. In one embodiment, the user output is delivered in a non-quantitative fashion, such as on the display device 32 of a portable device 30. For example, the user may be presented with one of the following results: “protein levels high, see a doctor,” “no protein detected,” “blood detected in urine, see a doctor,” “no blood detected,” “error in measurement,” and/or “please proceed to make the next measurement.”

As discussed, the collector 42 or container 48 can be either pressed up against or placed near the front sensor panel (e.g., the lens 14) of the unit 12, or inserted into the insertion portion 40 within the unit 12 for transmission in fluorescence mode experiments. Based upon the configuration of the imaging sensor 18 and the light source 24, the unit 12 may operate in a back-scattering mode, reflectance mode, and/or a transmission mode. The mode is dependent upon the relative positioning and spacing between the imaging sensor 18 and the light source 24.

With respect to another preferred and non-limiting embodiment, it is recognized that the measurements can be performed by unskilled users at the point-of-care. Some of these techniques may be compatible with existing urine collectors 42 or containers 48, and hyperspectral measurements may be made in an open top of the collector 42 or container 48. In one preferred and non-limiting embodiment, transmission and fluorescence experiments require an appropriately transparent collector 42 or container 48. As discussed, and since high protein levels are indicative of preeclampsia, acute kidney failure, or other renal problems, the system 10, whether using a display device 32 of the unit 12, or in some other display device in the system 10, issues a clearly-recognizable alert if levels of protein are above normal. In addition, the system 10 may measure the ratio of protein to a reference analyte, such as creatinine. Still further, the optics of the unit 12 can be arranged in various configurations in order to provide the appropriate light sample through the lens 14, filter arrangement 16, and to the imaging sensor 18. This permits making accurate absorbtance, reflectance, scattering, and/or fluorescence measurements in various fluids, such as urine.

With further reference to the preferred and non-limiting embodiments of FIG. 6, and in place of the collector 42 or container 48 described above, the test strip 44 is used. Accordingly, the hyperspectral imaging system 10 and hyperspectral imaging unit 12 is in the form of a test strip 44 readout system, which may be used to detect the presence or level of a particular molecule or class of molecules in various fluids that are brought into contact with the test strip 44. As discussed above, the test strip 44 may be manufactured to contain certain chemicals that cause a change in the spectral characteristic of parts of the test strip 44 due to chemical interaction with particular molecules or classes of molecules. As is known, existing test strips, e.g., pH test strips for measuring acidity or pregnancy test strips, typically cause a color change that is visible to the human eye. By instead using the hyperspectral imaging system 10 and unit 12 of the present invention to take an image of the test strip 44, reagents on the test strip 44 can be used that do not cause such a color change that is observable to the human eye. For reagents that cause a color change in the visible light spectrum, and by analyzing the spectral characteristics instead of the composite color, increased accuracy of the measurement can be achieved over both analysis with the human eye, or over images taken of the test strip 44 using a conventional monochromatic or color imaging system.

As discussed above, one or more light sources 24 can be used in this preferred and non-limiting embodiment of the unit 12, and these light sources 24 may be white, with either a continuous spectrum, or a specific spectrum, where only specific spectral bands have a high level of emissivity. Various combinations of such light sources 24 can be active for successive hyperspectral image captures of the same test strip 44. As discussed above, these light sources 24 can include incandescent bulbs, light emitting diodes, laser elements, chemical elements, and the like.

In another preferred and non-limiting embodiment, the test strip 44 can be imaged under similar or distinct illumination conditions over several time intervals. This allows capturing not only a fixed change in the spectral characteristics of reagents on the test strip 44, but also the variants of these spectral characteristics over time. The effectiveness, usefulness, and/or accuracy of the captured data and parameters are dependent upon the reagents used, as well as the speed at which they react with the test sample. In one preferred and non-limiting embodiment, the time interval and/or capturing process is implemented for several seconds to several minutes, in other embodiments, several hours to several days, or even several weeks to several months.

In one preferred and non-limiting embodiment, the test strip 44 is either immersed in the fluid F to be tested, or a sample of the fluid is applied to the test strip 44. The reagents on the test strip 44 are allowed to potentially react with compounds in the fluid for a given duration, e.g., about 60 seconds, and the reagents may change in color or in spectral or hyperspectral characteristics due to this chemical interaction with fluid compounds. The test strip 44 is illuminated, either using the ambient light, or by activation of the light sources 24 described above, and an image of the test strip 44 is acquired using the unit 12. It is envisioned that multiple such images are obtained, under the same illumination, or possibly by activating different light sources 24, which may have a different spectral profile. Taking multiple images under identical light conditions allows capturing varying spectral reflection characteristics that the reagent has on the test strip 44. This variation could be quantified, for example, in order to detect concentrations of analytes of interest in the test sample. By using illumination with different spectral profiles over multiple image captures of the same test strip 44, fluorescent response of the reagent can be measured.

The image processor 20 and/or the processor 22 processes the acquired images, and may display them on the display device 32 using false-color representation to highlight spectral ranges of interest, or through some automated image analysis process, in order to match the spectral profile of the reagents on the test strip 44 with the spectral curves of the reagent after interaction with a fluid compound of interest. As discussed above, this comparison will lead to biological data or biological condition information for use in a diagnostic process.

As discussed, the system 10 and unit 12 of the preferred and non-limiting embodiments of FIGS. 4-6 can be used to effectively capture and analyze hyperspectral images of collected urine and/or images of urine testing strips. With regard to the assessment of a whole urine specimen, normal urine does not contain cells, bacteria, crystals, or protein. The system 10 and unit 12 of this embodiment can be used for detecting changes in the urine hyperspectral signature generated by these products at different concentrations. To assess hyperspectral absorptivity of a urine sample, there is desirably a standard background, perfectly reflective and aligned, or hyperspectrally “white”, which may involve a double-pass with reflection or scattering behind the target medium TM or region of interest 130. If necessary, to calibrate the system 10 and/or unit 12, a library of normal urine sample images may be collected and used for each potential user of the unit 12, such as data and information stored or provided in the database 26.

As also discussed above, the urine test strips 44 may be used to evaluate multiple urine parameters, including glucose, protein, pH, nitrites, ketones, bilirubin (urobilinogen), and specific gravity. Through impregnation of these test strips 44 with reagents that are capable of reacting with these analytes to produce specific patterns, the biological data and information can be obtained. This may include either a qualitative assessment, e.g., normal urine versus proteinuria, or a quantitative assessment, e.g., glucose levels in the urine. Analysis of such hyperspectral image data will allow for direct identification of urine contaminants. Additionally, urine specific gravity, which is a measurement of urine solute concentration/urine osmolality, may be detected through changes in urine color as a result of increased solute content and/or decreased water excretion, with resulting increased urine turbidity. Variations in urine osmolality may be correlated both with urine color and turbidity, and thus may be estimated through smaller changes in color indiscernible by the human eye.

In one preferred and non-limiting embodiment, the system 10 and unit 12 includes the above-discussed portable device 30 that includes an internal power supply and, optionally, a Bluetooth module, which facilitates transmission of hyperspectral image data from the portable device 30 to a cloud-spaced storage entity, e.g, the database 26. Of course, this portable device 30 may offer data transfer through wired connectivity. Thereafter, the hyperspectral image data, such as the datacubes, can then be processed and analyzed through customized analysis software to determine the biological data and/or biological condition information. By identifying the specific spectral signatures, or spectral signature window/ranges of abnormal urine products, the system 10 and unit 12 of the present invention will be capable of acting as a detector for abnormal urine products, thus replacing the more laborious microscopic urine assessment.

The hyperspectral image data, such as the data captured through any of the systems 10 and units 12 described above or below, may be stored within the unit 12 (e.g., the portable device 30), processed within the unit 12, and/or sent to a remote system or processor 22 for storage and subsequent processing. Such remote storage and processing may occur through a variety of connectivity architectures. Of course, the hyperspectral imaging unit 12 may act as a standalone computer for both image capture and processing and/or to forward captured hyperspectral image data for external storage and/or processing non-wirelessly. Long-term storage will allow other users or parties to access previously-collected urinalysis data. In addition, data collected from other hyperspectral imaging systems 10 and units 12 will allow correlation of hyperspectral imaging data for assisting in diagnosis and determination of biological data. It is further envisioned that any of this hyperspectral image data, light information, biological data, biological condition information, or any other data obtained by or generated by the system 10 and unit 12 can be stored in the “cloud” or elsewhere for analysis of spectral signatures, which can be correlated with the previously-recorded hyperspectral signatures in order to yield both a finding, e.g., quantification of components in the fluid F, and a diagnosis, e.g., hematuria. If collected by the patient, this data may be synchronized with either an electronic medical record to which the patient has access, or another cloud storage space from which other personnel or users are capable of pulling clinical data.

In one preferred and non-limiting embodiment, the hyperspectral imaging system 10 and hyperspectral imaging unit 12 facilitate the hyperspectral image capture of the target medium TM or region of interest 130 (e.g., a urine specimen) as determined by the user. With this image capture, a hyperspectral datacube can be generated, which contains hyperspectral data for individual image pixels across a continuous portion of the electromagnetic spectrum, e.g., 400 nanometers to 1,700 nanometers. Both the pixel density and the electromagnetic spectrum from which the unit 12 is capable of sensing may be modified or otherwise configured. In addition, information can be displayed on the display device 32 (or other displays or screens) to provide the user with information, e.g., changes in the hyperspectral signature from normal, uncontaminated urine. For example, increased signal within a window of the hyperspectral spectrum may indicate the presence of red blood cells or white blood cells. Alternatively, a nephrotic patient with proteinuria will have a visibly different urine appearance. In some cases, the hyperspectral signature of the contaminated urine may be predicted based on the signature of these contaminants and other clinical settings. For example, the wavelengths used to identify hemoglobin visualized through skin range from 542 nanometers to 548 nanometers in some studies indicate a potential window for identifying red blood cells in urine and diagnosing microhematuria, which is not evident to the naked eye.

In another preferred and non-limiting embodiment, the diagnosis produced by the system 10 and unit 12 may be combined with other imaging data stored in the original device and/or with subjective information obtained from the user through a diagnostic aid interface. This additional information may be used in concert with hyperspectral image data and/or biological data to frame recommendations for the user or patient based on the findings generated across all of the equipment.

As illustrated in the preferred and non-limiting embodiment of FIG. 6, the hyperspectral imaging unit 12 is equipped with the lens 14 for taking a hyperspectral image of the test strip 44. This image can be taken under illumination of the light source 24, and the unit 12 is connected using a data link 58 with a processor 22 (e.g., a computer system, a microcontroller, and/or an automated data processing system). The data link 58 is used for transferring hyperspectral images from the unit 12 to the processor 22, and may also be used for controlling the unit 12. The light source 24 may also be controlled by the same processor 22, such as through a digital or analog interface 60. The hyperspectral images may then be stored in the database 26 or some other data storage system. Alternatively, the information and data can be transmitted and stored in a variety of different databases. Further, as discussed above, some or all of these components, i.e., the lens 14, data link 58, processor 22, light source 24, database 26, and digital or analog interface, may all be provided in an integrated hyperspectral imaging unit 12. Accordingly, the analysis and determination of biological data and information can be done within the portable device 30 (in a local environment) or by some remote processor 22 (in a remote environment).

Lateral flow tests are known and used to analyze a wide range of specimens, including urine, saliva, serum, blood, and the like. As is well known in the art, to perform the diagnostic, the lateral flow test is placed in the specimen for a specified amount of time, and such test results are normally read manually by looking at a test strip, change in color on the test strip, and/or comparing the visually-assessed color with a standardized color palette under appropriate illumination conditions. Lateral flow assays are used in medical offices as a rapid test for ailments, such as urinalysis, strep throat detection, influenza detection, and the like. Each test normally has two indicator areas, one of which is the “control” area and the other the “test” area. When the diagnostic is performed, the control area should always change color, verifying that the diagnostic was used correctly, while the test indicator only changes color if the analyte is present. For instance, with a strep throat lateral flow assay, a person with a strep throat infection would see both the control and test areas change color. In addition, multiple tests are often performed in parallel, such as a positive control test and a negative control test, to verify that the test strips are properly working, five from contamination, and to raise confidence in the avoidance of false positives and false negatives.

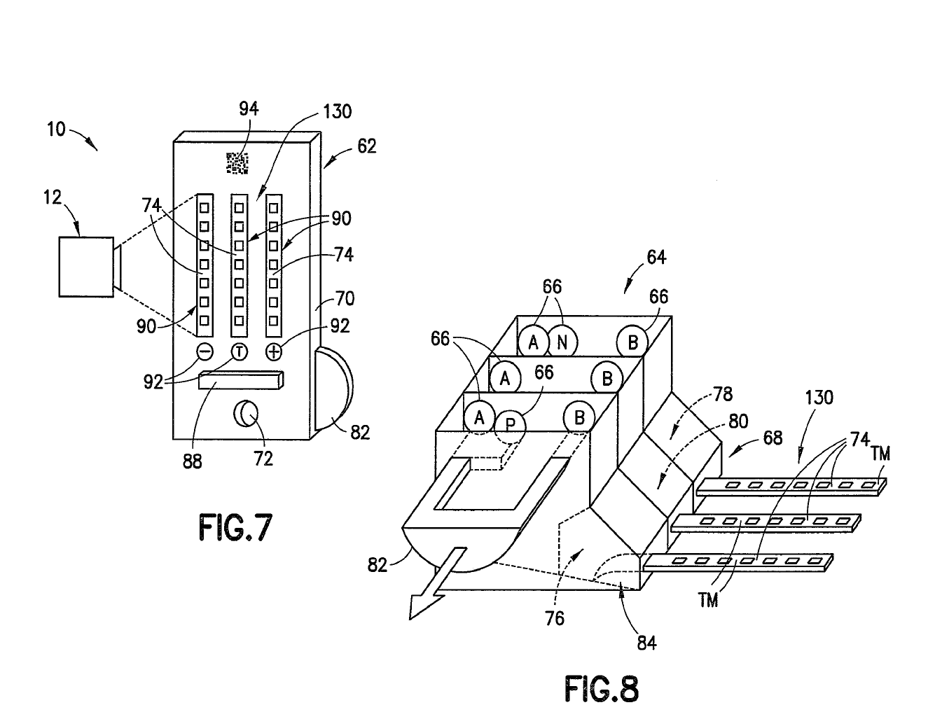

In one preferred and non-limiting embodiment, and as illustrated in FIGS. 7-9, the hyperspectral imaging system 10 includes a lateral flow assay testing device 62 for use in detection and quantification of analytes in samples, such as a saliva sample containing cells and fluid. As discussed in more detail hereinafter, the system 10 and testing device 62 and system 10 include the appropriate testing materials to facilitate the detection of an analyte in a sample containing certain compounds, such as whole cells, blood, saliva, urine, and the like. In particular, the system 10 and testing device 62 provide a medical test for lateral flow assays and, in one preferred and non-limiting embodiment, the testing device 62 includes the reagents integrated or provided on or with the testing device 62. Still further, after the test is performed, the results are captured using any of the above-described hyperspectral imaging units 12 for obtaining the appropriate hyperspectral image data.

In another preferred and non-limiting embodiment, and as best illustrated in FIG. 8, a reagent cartridge 64 is provided and includes one or more reagent pouches 66 that contain a specific reagent chemical, e.g., reagent A, reagent B, and reagent P. These pouches 66 are configured to be opened or disturbed, such that the reagent chemical flows into a mixing chamber 68. In addition, the testing device 62 includes a housing 70 with an opening 72 configured to receive a fluid sample, and the opening 72 is in fluid communication with one of a plurality of mixing chambers 68. Still further, each mixing chamber 68 is in fluid communication or capable of being in fluid communication with a test strip 74. In another preferred and non-limiting embodiment, the testing device 62 includes three mixing chambers 68, namely a positive mixing chamber 76 containing or configured to obtain a positive indicator (i.e., reagent P) of a specified biological condition, a negative mixing chamber 78 containing or configured to obtain a negative indicator (i.e., reagent N), and a sample mixing chamber 80 configured to receive the fluid sample via the opening 72.

The system 10 and testing device 62 illustrated in FIGS. 7-9 can be used in a variety of situations and applications, including home testing, point-of-care testing, and/or laboratory use, such that the testing device 62 can be shipped, stored, and used in a range of environmental conditions. As discussed, the chemical reagents, e.g., reagent A, reagent B, reagent P, and reagent N, are encapsulated to avoid evaporation and leakage. In one exemplary embodiment, reagent A and reagent B are stored separately and each contained in the above-discussed pouch 66, which may be in the form of a small aluminum foil pouch or plastic pouch, and potentially in spherical form. It is envisioned that such reagents can be stored in the pouches for up to 10 years. In one preferred and non-limiting embodiment, the materials used to create these pouches 66 include thin aluminum foil (approximately 0.5 millimeters thick) or waterproof plastic, such as polyethylene plastics or mylar (approximately 0.5 millimeters thick). To initiate the test, these pouches 66 are mechanically burst by pulling on a tab 82, and either through tearing or compression, the chemical reagents are released from the pouches 66 into the respective mixing chambers 68. For each test assay, one pouch 66 of each chemical reagent A and B is used. In addition, and for providing a positive control in the positive mixing chamber 76, reagent P (also in a pouch 66) is used. Similarly, and for providing a negative control in the negative mixing chamber 78, reagent N (also in a pouch 66) is used. In particular, reagent P is the “dummy” reagent meant to trigger the test and reagent N is the “dummy” reagent meant to provide a negative or standard calibration reference. Again the pouches 66 containing these reagents P, N are capable of being compressed, torn, burst, or otherwise opened using the above-discussed tab 82.

As the reagents are separated prior to mixing, and therefore must be properly mixed prior to the lateral flow assay, as discussed, the tab 82 is used to release these reagents A, B, and P, into the respective mixing chambers 68. In one preferred and non-limiting embodiment, and as best illustrated in FIG. 8, the pouches 66 are slightly elevated above each respective mixing chamber 68, such that when they are opened, they flow downward with the help of gravity into the separate mixing chambers 68. It is further envisioned that the pouches 66 are positioned such that, when opened or burst, the chemical reagent contacts a wall or other surface of the mixing chamber 68 and allows for the appropriate mixing of the reagents in the mixing chamber 68. Of course, it is also envisioned that the testing device 62 can be swirled or shaken, or some other manual or automated mixing arrangement can be used. By using the elevated positioning of the pouches 66, the flow of the reagents is unidirectional towards each respective mixing chamber 68.

With continued reference to FIGS. 7-9, and in this preferred and non-limiting embodiment, the mixing chamber 68 may be designed and shaped like a small reservoir or watering hole, where the contents from the pouches 66 flow. Accordingly, these mixing chambers may also have slanted or sloped walls or floors to direct the mixed reagents towards one end 84 of each mixing chamber 68. It is at this end 84 of each mixing chamber 68 that a portion of the test strip 74 is positioned or positionable. It is further envisioned that the positive control chemical reagent P may be positioned in or otherwise located in or near this end 84 of the positive mixing chamber 76. Accordingly, the tab 82 (or a separate tab) may be used to release this positive chemical reagent B into any of the appropriate portions of the positive mixing chambers 76, such as at the end 84 of the positive mixing chamber 76. Further, the opening 72 allows access to the sample mixing chamber 80, such that when the liquid sample is deposited or placed into the opening 72, it flows towards the end 84 of the sample mixing chamber 80.

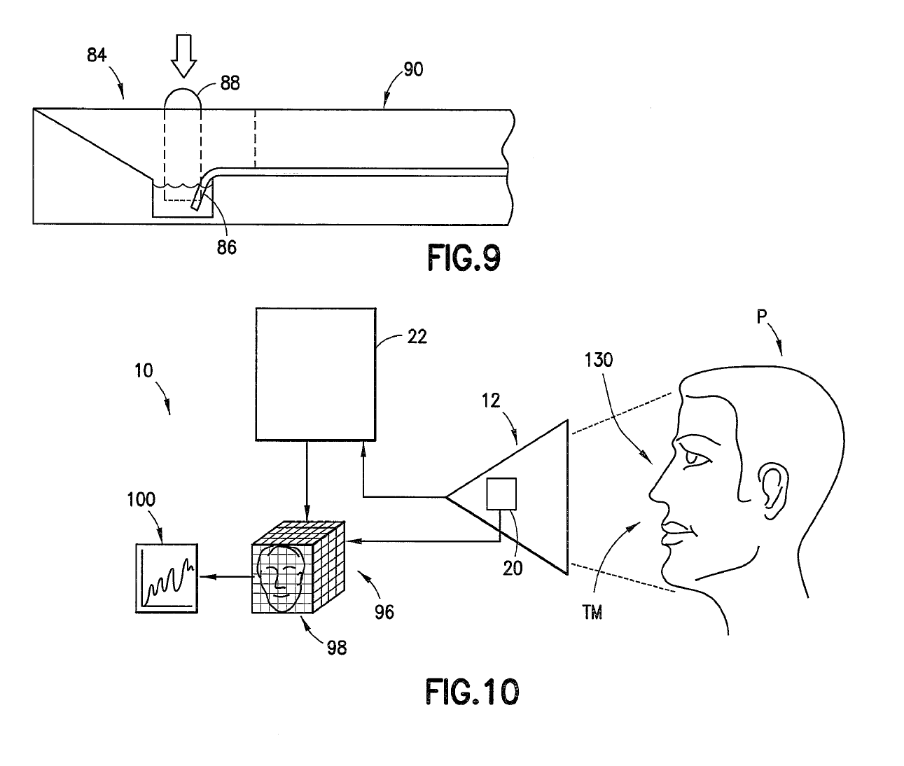

Depending upon what pathogen is the object of detection, the test strips 74 will include the appropriate chemical indicators of that specific disease for the specified testing device 62. In another preferred and non-limiting embodiment, and as illustrated in FIG. 9, an end 86 of each test strip 74 may be positioned just above each respective mixing chamber 68. When the test is ready to be performed, each end 86 of each test strip 74 will be urged into the respective mixing chamber 68 by pressing a button 88 on the housing 70. In particular, by pressing this button 88, the end 86 of each test strip 74 is pushed into the mixed reagents in the mixing chamber 68. The liquid will then climb the test strip 74 based upon capillary action.

In order to perform the analysis, a hyperspectral imaging unit 12 obtains hyperspectral image data from each test strip 74, such as through a respective window 90 on the face of the housing 70. Further, each window 90 will include or be associated with an indicator 92, such that the tester can understand which mixing chamber 68 is being tested. In this embodiment, a “−” symbol is used as the indicator 92 for the negative mixing chamber 78 (and results), a “T” is used as the indicator 92 for the sample mixing chamber 80 (and results), and a “+” symbol is used as the indicator 92 for the positive mixing chamber 76 (and results). In addition, the hyperspectral imaging unit 12 of this preferred and non-limiting embodiment is configured to identify the testing device 62, its orientation, and its distance from the unit 12. In one preferred and non-limiting embodiment, a data element 94, such as a Quick Response (QR) code (standardized in ISO/IEC 18004:2006), is printed or otherwise applied on each testing device 62. As is known, such a QR code provides the ability to encode data and orientation information for use with each individual testing device 62. In addition, this QR code will be used to compute the unique identity, e.g., serial number, date of manufacture, place of manufacture, and the like, and orientation of the testing device 62 relative to the hyperspectral imaging unit 12 for determining the size of the result and angle of the testing process. In one preferred and non-limiting embodiment, the user need only take a hyperspectral image of the face of the testing device 62, and as long as the data element 94 is visible with sufficient resolution, the results of the test will be correctly calculated.

In another preferred and non-limiting embodiment, and as illustrated in FIG. 10, the target medium TM or region of interest 130 is at least a portion of a person's face, and the biological data are in the form of biological information that is determined based upon the hyperspectral features of at least a portion of the person's face. In particular, since a hyperspectral image contains a full spectral profile of each pixel, the image can be used to detect the blood volume pulse by measuring how the scattering, reflectance, or transmission of one or multiple wavelengths of light change over time. When a heart beats, blood is pumped throughout the body. When a person has been exercising, it is easy to notice the color change in their face when the blood rushes to the surface of the skin; but this color change is happening constantly every time the heart beats. This color change is subtle, and often not noticeable to the naked eye. However, by using the hyperspectral imaging system 10 and hyperspectral imaging unit 12 of the present invention, such subtle changes in color can be easily identified. In particular, by recording specific spectral wavelengths from the region of interest 130 or the target medium TM, such as the face or skin of a person P, the blood volume pulse waveforms of the person P can be extracted from the signal. The signal can be processed, including detrending and normalization, to measure heart rate, respiratory rate, and heart rate variability. In addition, this processing method may include averaging the pixels across relevant wavelengths within the region of interest 130.

With continued reference to FIG. 10, and in one preferred and non-limiting embodiment, the hyperspectral imaging system 10 and hyperspectral imaging unit 12 could be used to measure physiological signs from a target medium TM representing the person's face, such as the skin or other biological features. Once the target medium TM or region of interest 130 is determined, a sequence of images or video, i.e., hyperspectral images, are captured and time-stamped. In one preferred and non-limiting embodiment, a datacube 96 is created (as discussed in detail above), and includes an array of pixels 98. The source for each pixel 98 of the image includes data about a wide spectrum (e.g., 400 nanometers to 1,700 nanometers) of light. Thereafter, spectral information 100 for each pixel 98 in the image is determined directly from the source without decomposition. By selecting a suitable sub-range of wavelengths from the spectral information 100 in each pixel 98 in the image, a signal for the blood pulse volume can be extracted.

In this preferred and non-limiting embodiment, and as discussed above, the hyperspectral imaging system 10 or hyperspectral imaging unit 12 is configured to divide a sample spectrum into tens, hundreds, or even thousands of bands or spectral information 100, typically with a ten nanometer bandwidth or less. This higher resolution spectrum hyperspectral data can be added together within each pixel 98, as in RGB-based systems, but with better color resolution, to produce a more perfectly resolved “overall” color at each pixel 98. Alternatively, the full spectral data or spectral information 100 can be observed at each pixel 98, resulting in a maximal picture of surface scattering, absorbtance, and/or reflectance for the target medium TM or region of interest 130, such as the human face or other skin surface.

Since the hyperspectral imaging unit 12 allows the direct monitoring of small ranges of wavelengths, this can result in a more accurate measurement of physiological parameters from a simple sequence of images or video. The physiological measurements could then be reviewed by the subject being monitored, or remotely by a trained staff, such as doctors, nurses, healthcare workers, roommates, police, loved ones, etc. These readings can then be used to make health decisions, such as a suggestion to exercise, or whether a patient should be monitored more closely or perform an examination with a doctor. This process can also be used to enable the accurate non-contact monitoring of physiological parameters, without the need for separate devices, such as a pulse oximeter finger clip or chest strap. The person being monitored would therefore not be encumbered by these physical accessories, and instead the data would be gathered passively.

In one preferred and non-limiting embodiment, the hyperspectral imaging unit 12 can be used in connection with, connected to, or otherwise integrated with a television, a tele-screen, a computer, and the like, and be configured to passively record data. This could include the physiological measurements of a person near the device, such as a person sitting in a chair or simply walking by. One potential implementation would allow for the monitoring of persons in the home, in the working environment, or in any indoor/outdoor environment, in order to gather information to make individual or general health decisions using the data gathered from a large number of people.

Accordingly, and with continued reference to FIG. 10, the hyperspectral imaging system 10 and hyperspectral imaging unit 12 provide a method for measuring physiological parameters by: capturing a sequence of images (or video) together with the associated hyperspectral data over a wide spectral range, with a source signal from each subset within this range available without decomposition; identifying a location or multiple locations anywhere on a person's body in a frame of the captured image; establishing a region of interest 130 (i.e., on the target medium TM); and capturing the data to determine the biological condition, such as a physiological parameter. In addition, spatial averaging can be used over the region of interest 130 or target medium TM within a given spectral subset, and it is envisioned that simultaneously physiological measurements can be obtained. Still further, and with continued reference to the preferred and non-limiting embodiment of FIG. 10, the hyperspectral imaging system 10 and hyperspectral imaging unit 12 can be used for remote real-time face detection, as well as facial tracking over time. Such a process is useful and based upon the vast person-to-person spectral variability for different tissue types.

In one preferred and non-limiting embodiment, hyperspectral imaging unit 12 is in the form of a hyperspectral camera, which captures an image of the person P and then locates the facial region based on previously-determined spectral characteristics. After the hyperspectral image is taken, the hyperspectral image data are processed in real-time using software to locate and outline the facial region in the captured image. In this manner, the system 10 and unit 12 (and associated methods) enable superior spectral resolution over conventional, digital image-captured technologies across the visible spectrum. Since, as discussed above, and illustrated in FIG. 10, the datacube 96 includes a full spectral profile of the pixels 98, with resulting spectral information 100, the image can be used to detect and locate the outline of a face by identifying spectral signatures of human skin, hemoglobin, hair, and the like. In particular, the epidermal and dermal layers of human skin constitute a scattering medium that contains several pigments, such as melanin, hemoglobin, bilirubin, and beta-carotene. Small changes in the distribution of these pigments induce significant changes in the skin's spectral signature. The effects are large enough to enable for the automated separation of melanin and hemoglobin from hyperspectral images. Accordingly, and using the above-described system 10 and unit 12, full spectral data can be observed at each pixel 98 resulting in an enhanced and resolved picture of the surface scattering, absorbtance, and/or reflectance for the target medium TM or region of interest 130. As discussed, each pixel 98 exhibits specific spectral information 100 (or spectral signature), which can be matched to predetermine spectral signatures of the face, as well as the outline of the face, with the corresponding signatures correlated by their pixel positioning in the image. An outline may then be drawn in the image where the face outline spectral signatures occur.

In order to accomplish facial recognition and/or facial identification, the system 10 can determine specific spectral signatures of the face of each person P, and then save these spectral signatures, along with the input of the person's name or other identification, into the database 26. The spectral signature can then be retrieved and matched to a facial spectral signature of the person P determined at a later date when a subsequent image of the person's face is recorded in the future. Again, based upon the nature of the above-described hyperspectral imaging unit 12, the direct monitoring of small ranges of wavelengths allow for the more accurate identification and recognition of a person's face (or other parts of the body).

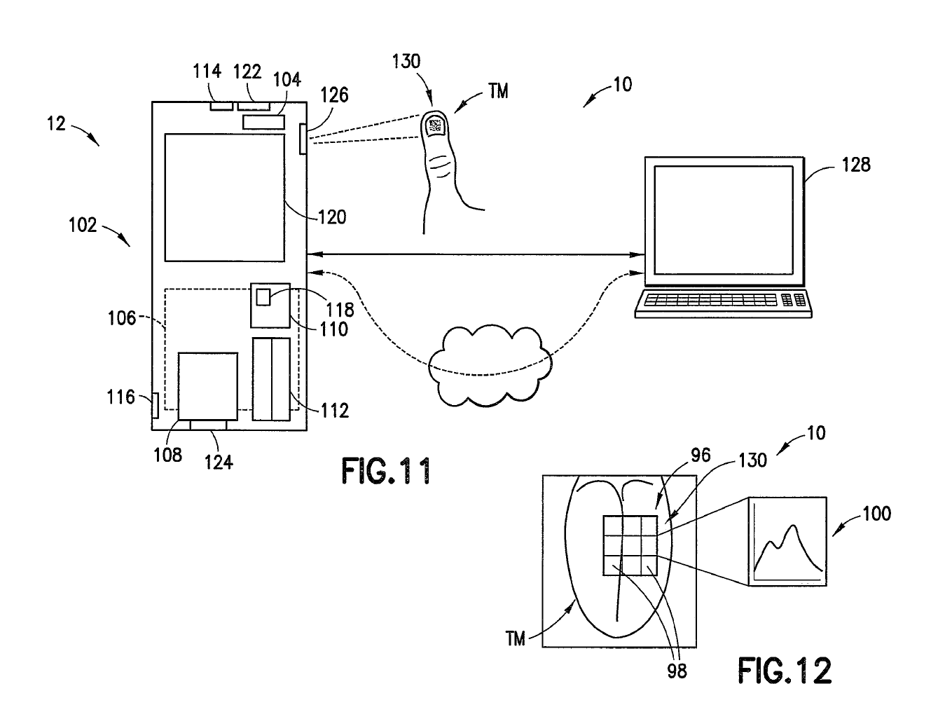

In another preferred and non-limiting embodiment, and as illustrated in FIG. 11, the hyperspectral imaging system 10 and hyperspectral imaging unit 12 are used in determining biological condition information or other biological data associated with a fungal species. Fungi are eukaryotic organisms that reproduce by releasing spores. Fungi can be grown in any environment that is moist and warm, including older buildings, outdoors, and inside the body. Fungi are used by humans in a number of ways, including as food and drug sources. Fungi also serve as a health hazard, causing disease to humans through infection, allergies, or reaction to mycotoxins released by fungi.

The most common methods of fungus identification involve culturing fungal samples and performing microscopy or using molecular-based assays. Increasingly, polymerase chain reaction (PCR) techniques for DNA-based identification are used. Commercially-available kits exist that allow consumers to submit a sample for analysis at a cost of $15.00-$200.00 per instance. Current methods for identification of fungal species require sampling of the fungus, lab preparation, and analysis by experts. In all cases, samples must be analyzed by trained mycologists whereupon the process is costly, labor intensive, slow, and subject to human error. Electromagnetic radiation has been used to detect fungus, and hyperspectral methods have been developed to identify fungal species. However, this method requires sample extraction, preparation, and analysis by a mycologist; and, although faster than previous techniques, this method poses similar challenges as previous technologies.

In the preferred and non-limiting embodiment of FIG. 11, the hyperspectral imaging system 10 and hyperspectral imaging unit 12 are used for the identification of fungal species (such as bacterial fungal infections, mold, yeast, mushrooms, and the like), which does not require contact with the sample, preparation of a culture in a laboratory, or analysis by a mycologist. In this embodiment, the unit 12 is a portable device 102 that includes any suitable and/or desirable type of hyperspectral imaging sensor 104, which can be used to capture hyperspectral image data for a target medium TM or region of interest 130, such as an area on a person, plant, or animal that is suspected of being or including a fungus.

In one preferred and non-limiting embodiment, the portable device 102 (e.g., a smartphone or other handheld device) includes an encased computer 106 with a battery 108, CPU 110, memory 112, the hyperspectral imager sensor 104, an optional light source 114, a data port 116, and a wired and/or wireless communication chip 118 (e.g., Ethernet, WiFi, Bluetooth®, 4G, and the like). A viewfinder window 120 (of a visual display) is located on the front of the device 102, and a lens 122 is located on the device 102. In this embodiment, the light source 114 is positioned on or near the lens 122, and the embedded hyperspectral imaging sensor 104 is positioned directly behind lens 122. An on/off button 124 is located on the bottom of the device, and the battery 108 inside of the casing is connected to on/off button 124. A button 126 that the user presses to take an image is located on the side of the device 102. Of course, it should be recognized that these are only exemplary layouts and positioning of the various components of the portable device 102.

In operation, a hyperspectral image of the target medium TM or region of interest 130 is taken through the lens 122, such that the device 102 is positioned by the user so that the line of sight from the device 102 to the region of interest 130 is clear. The device 102 is then powered “on” by the user by engaging the on/off button 124 (assuming the device 102 is in a powered-off state). Next, the user engages button 126, whereupon the light source 114 illuminates the target medium TM and the hyperspectral imaging sensor 104 is caused to generate a hyperspectral image preview of the target medium TM or region of interest 130, which is displayed on viewfinder window 120 on the front of device 102. When the user presses the button 126 a second time, the hyperspectral imaging sensor 104 captures and/or generates the hyperspectral data, e.g., one or more hyperspectral images, and the hyperspectral image data corresponding to the captured hyperspectral image is stored in the memory 112 for later retrieval. Alternatively, a single engagement of the button 126 can cause hyperspectral imaging sensor 124 to immediately capture and/or generate the hyperspectral data and/or hyperspectral image data, and store the data in the memory 112. The hyperspectral image data stored in the memory 112 may be transmitted, either via a wired connection or wirelessly, from the device 102 to a remote data storage system 128, which may be in the cloud, via the communication chip 118 of the device 102.

After or during transmission, the transmitted hyperspectral image data may be analyzed, after which the name and defining characteristics of the fungal sample can be relayed back to the device 102 and displayed in viewfinder window 120. Such analysis may also occur remotely by third-party analytics or software programs. It is further envisioned that the portable device 102 may include video capabilities, positioning systems, or any other hardware, firmware, or software to further augment the overall functionality of the device 102. In one preferred and non-limiting embodiment, the portable device 102 has similar dimensions to a smartphone. Alternatively, some or all of the above-described functions and operations can be performed on a smartphone having the described hyperspectral capabilities integrated thereon. Similarly, the portable device 102 may be in the form of any of the above-described hyperspectral imaging units 12, which may be controlled or controllable using the data storage system 128, such as a personal computer or the like. In such an embodiment, any of the biological data, e.g., the fungal information or data, can be displayed on the monitor of the personal computer.

In a still further preferred and non-limiting embodiment, and as illustrated in FIG. 12, the hyperspectral imaging system 10 and hyperspectral imaging unit 12 are used in connection with tongue T diagnosis, such that the target medium TM or region of interest 130 is at least a portion of a person's tongue. Accordingly, the biological data in this embodiment are in the form of biological condition information at least partially determined based upon the hyperspectral features of the tongue T. As is known, the tongue is a vital indicator of human health, and has been used for diagnosis in various situations. Features of interest include tongue color, tongue fissures or cracks, sublingual veins, tongue coating, and the like. Historically, the process of tongue diagnosis has been subjective, but the rapid progression of information technology affords opportunities to use computerized image analysis for tongue diagnosis. However, such existing technologies are deficient as it is difficult to distinguish between the tongue and neighboring tissues, as well as between tongue coating and tongue substance. However, and as illustrated in FIG. 12, in this preferred and non-limiting embodiment, the system 10 and unit 12 enable superior spectral resolution over conventional, digital image-capture technologies.

As discussed above in connection with, for example, the embodiment of FIG. 10, the system 10 and unit 12 of this embodiment is configured to divide a sample spectrum into tens, hundreds, or even thousands of bands, and these higher resolution spectral hyperspectral data can be added together within each pixel 98 as provided from the datacube 96. Alternatively, the full spectral data can be observed at each pixel 98 for providing the spectral information 100 and providing the best “picture” of surface scattering, absorbtance, and/or reflectance for the target medium TM or region of interest 130 on the target medium TM, such as the tongue T. Again, the datacube 96 representing the spectral characteristics of the target medium TM or region of interest can be subject to image extraction techniques, such as support vector machines or spectral angle mapping, wherein the tongue T is differentiated from the surrounding tissues. Once the tongue T has been isolated, the number of important spectral features in the tongue T can be quantified and compared against disease states. Important features, including tongue color, tongue fissures or cracks, sublingual veins, tongue coating, and the like, can be extracted from the hyperspectral datacube 96 of the isolated tongue T. While using conventional RGB imaging for matching tongue color images is available, it is difficult to mitigate color distortion of tongue images based on inconsistent lighting conditions. Conversely, since the colors of the target medium TM or region of interest 130 closely relate to its spectrum, and since the spectra of an organism in the range of wavelengths of visible light (approximately 400 nanometers to 750 nanometers) completely includes the RGB color space, spectra can be used to identify tongue colors more accurately. Accordingly, and by using the system 10 and unit 12 of the present invention, it is possible to examine a target medium TM or region of interest 130, a datacube 96, or a pixel 98 of an isolated tongue T and determine its color based on its spectral signature.

Tongue crack extraction and classification is another area that has been investigated using conventional RGB imaging, which is also improved through the use of the presently-invented system 10 and unit 12. There are typically three steps to tongue crack extraction, which are finding, tracking, and linking, and this process leads to the classification of the cracks into one of 16 typical tongue-crack categories. The hyperspectral datacube 96 of the isolated tongue can be processed through an algorithm to reveal various tongue-cracking categories or classifications, such as through a comparison against existing hyperspectral references or other biological information or conditions in the database 26.

Further, and with continued reference to the preferred and non-limiting embodiment of FIG. 12, the hyperspectral imaging system 10 and hyperspectral imaging unit 12 can be used in the sublingual vein extraction and interpretation area. Extracting the sublingual vein from the surrounding tissues is complicated based upon the variability and thickness of the sublingual mucosa. The extraction process is improved by using the spatial and spectral data of the system 10 and unit 12. There are typically two types of quantitative features that are diagnostically meaningful after extraction of the sublingual vein image from the surrounding tissues, namely the breadth feature and the chromatic feature. In operation, an image of the person's open mouth is captured using the hyperspectral imaging unit 12, which creates a hyperspectral datacube 96 of the open mouth. From this datacube 96, the sublingual vein portion can be extracted using certain techniques, such as a spectral angle mapper or an improved spectral angle mapper. Using both spatial and spectral data, the breadth and chromatic features of the sublingual tongue can be characterized. Thereafter, some or all of these extracted features can be associated with disease states that are known or included in the database 26 or some other third-party reference or database.

In another preferred and non-limiting embodiment, the hyperspectral imaging system 10 and hyperspectral imaging unit 12 according to the present invention is used to determine biological data and/or biological condition information relating to a rash, a burn, a lesion, an inflammation, an allergic reaction, acne, a wound, a bruise, a skin condition, a dermatological condition, a symmetric condition, a diametric condition, an irregularity condition, a color condition, a size condition, and/or a depth condition. Accordingly, these conditions relate to biological conditions of the human body, primarily the skin. For example, rashes, burns, and lesions are normally evaluated based on an unenhanced visual or naked-eye analysis by a dermatologist, or an RGB camera system. These two methods are limited by a phenomenon called metamerism, where the illuminant on the lesion affects the appearance of that lesion. For instance, viewing a lesion outside in the sunlight versus inside under fluorescent light may radically alter the appearance of the lesion, and result in misdiagnosis or misunderstanding. One technique to determine a more accurate assessment of rashes, burns, and lesions is to use the system 10 and unit 12 according to the present invention. In particular, and since a hyperspectral image contains the full spectral profile at each pixel, the image can be used to detect and locate the presence and quantity of particular molecules within the skin. Examples of molecules that may be visualized in the skin include, but are not limited to, oxyhemoglobin, deoxyhemoglobin, and melanin. Locating the presence and quantity of molecules within the skin has a wide range of medical applications, including tracking the progress of wound healing or the extent of bruising.

According to this preferred and non-limiting embodiment, the hyperspectral imaging system 10 and hyperspectral imaging unit 12 obtain hyperspectral image measurements by first identifying the location of the rash, burn, or lesion in a captured image (or series of images) and establishing the region of interest 130 on the target medium TM, in the image. As discussed above, the source for each pixel of the image includes data about a wide spectrum of light, and the spectral information in each pixel in the images is available directly from the source without decomposition. To identify and quantify a rash or lesion, hyperspectral data are first captured by identifying the target medium TM or region of interest 130 of the skin in each frame of the captured hyperspectral images. With reference to the database 26, which may include rashes, lesions, and healthy skin images, the captured image will be processed to obtain useful signals, such as through principal component analysis, and attempt to match the spectral profile to the profiles in the database 26. Of course, this can be combined with known, normal imaging techniques, such as edge detection and/or shape matching algorithms to enhance identification and quantification. As discussed above, since the hyperspectral imaging unit 12 according to the present invention allows for the direct monitoring of small ranges of wavelengths, this information and hyperspectral data can be used in an automated identification process and/or quantification from an image or simple sequence of images or video. Of course, and as discussed above, it is recognized that the images may also be reviewed by a medical professional trained in hyperspectral imaging to finalize the diagnosis. This identification and quantification can then be used to make health decisions, such as whether a rash is contagious or not (e.g., chicken pox on a child), or requires a certain type of treatment (e.g., poison ivy on the arm) or immediate medical care (e.g., third degree burn on the hand).

In another preferred and non-limiting embodiment, the hyperspectral imaging system 10 and hyperspectral imaging unit 12 is used in connection with allergy monitoring and testing. Allergy tests normally involve taking a skin test to determine which substances cause an allergic reaction. Some of the allergens tested may include nickel sulfate, wood alcohols, potassium dichromate, and black rubber mix, amongst others. In the case of a patch test, a patch containing small independent amounts of one or more allergens is applied to the skin. After about 48 hours, the patch is removed. Based upon the reactivity of the skin to each allergen, the person's allergic response is determined by a physician by visual inspection when the patch is taken off, and may be repeated at certain intervals. In the case of a “prick” test, the process is similar, except as opposed to a patch, small independent amounts of one or more allergens are dropped on the skin and a scratch or needle prick is made at the area of contact to allow the solution to enter the skin. In conventional allergy tests, after a patch is removed, usually a physician visually checks the result of each test spot for swelling or allergic response. However, the system 10 and unit 12 according to the present invention can be used to obtain a quantitative, objective result of certain features, such as the presence of oxyhemoglobin, deoxyhemoglobin, and melanin. Locating the presence and quantity of molecules within the skin can be used to sense the presence of certain compounds or components, as well as mapping the size of spots and different grades of inflammation.

In operation, and according to a preferred and non-limiting embodiment, the system 10 and unit 12 can be used to measure the result of an allergy patch test or an allergy prick test using the hyperspectral imaging unit 12 to capture a hyperspectral image (or series of images) of the target medium TM or region of interest 130 on the skin corresponding with a patch or pricked area. As discussed above, after obtaining and capturing the hyperspectral data and analyzing and forming the datacube 96, the spectral information 100 for each pixel 98 in the image can be used to map the presence of melanin, proteins, swelling, discoloration, and the like. By using the system 10 and unit 12 according to this preferred and non-limiting embodiment, a more quantitative measurement of the result of each allergy test is provided.

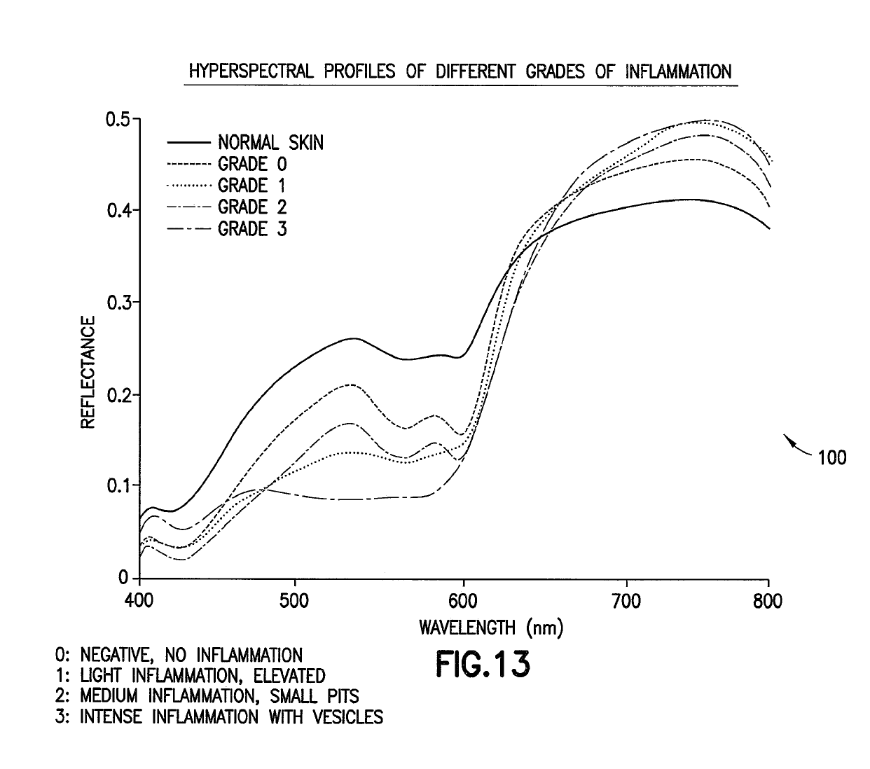

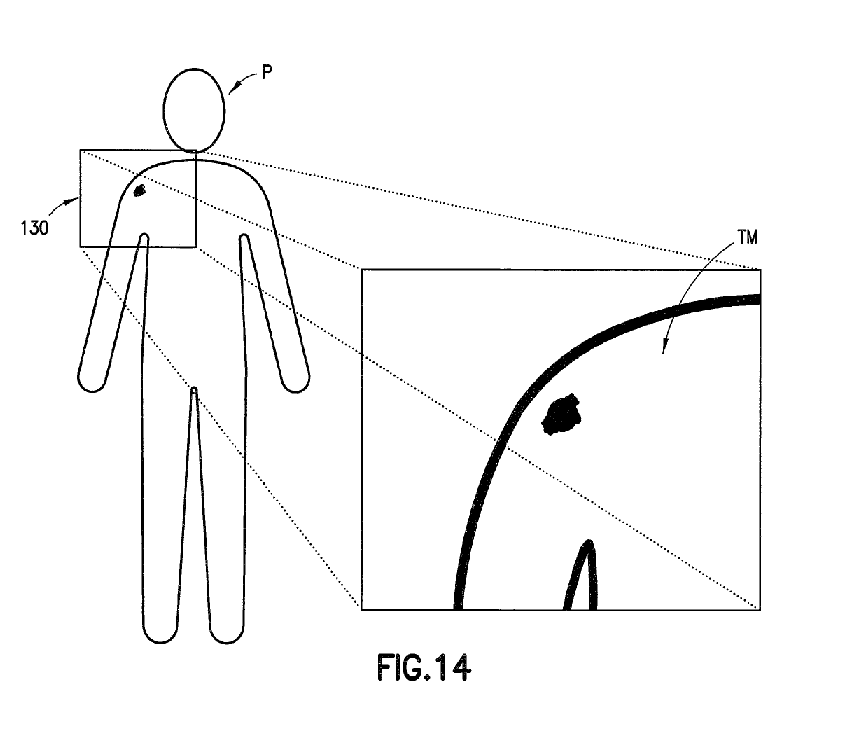

In one example, the biological data and/or biological condition information may be in the form of the spectral information 100 as illustrated in the graph of FIG. 13. In this example, the derived spectral information is assigned a numerical grade, such as grade 0, grade 1, grade 2, and grade 3 to each patch or prick result. Of course, these results could be separated even further, such as by grading from a scale from 0-10. The quantitative measurements captured by the hyperspectral image could then be reviewed by the subject being monitored, or remotely by trained medical staff, such as doctors, nurses, and/or health care staff. Still further, each grade could be associated with existing hyperspectral data or information, or other information and data derived from the database 26. In the example of FIG. 13, a grade 0 means that the skin has no inflammation; grade 1 means that the skin has light inflammation, and is elevated; grade 2 means that the skin has medium inflammation, and has small pits; and grade 3 means that the skin has intensive inflammation with vesicles.

Such grading and determination of associated biological data or information (or biological condition) can be used in connection with any of the target media TM or regions of interest 130 described above or below. Accordingly, the biological data and/or biological condition information for any of the embodiments in connection with the present invention can be displayed in raw, pre-processed, processed, and/or analyzed form, including, but not limited to, the datacube 96, information about the pixels 98, the spectral information 100, wavelength information, scattering, reflectance, and/or transmission information, grade or rank levels, textual condition information, graphical condition information, and the like. With respect to the present embodiment, the person can then make medical decisions based upon the outcome and the results displayed in FIG. 13, such as avoiding certain types of food (e.g., peanuts in the case of a peanut allergy), scents (e.g., in the case of sensitivity to certain perfumes), or certain work environments (e.g., in the case of sensitivity to allergens that might be found in nature, like tree sap).

Still further, and in accordance with the preferred and non-limiting embodiment discussed herein, the system 10 and unit 12 can be used to obtain a quantitative, objective result of hemometrical features where certain components can be visualized, e.g., oxyhemoglobin, deoxyhemoglobin, and melanin. Locating the presence and quantity of molecules within the skin has a wide range of medical applications, including tracking the progress of wound healing or the extent of bruising (as discussed above). Also, and as discussed, by scanning for molecules, as well as mapping the size of each spot, different grades of information can be applied, and after processing the spectral profile of each spot, can be matched to biological data or biological condition information in the database 26. Accordingly, the presently-invented system 10 and unit 12 can be used to quantify the result of each allergen spot test to a degree that the naked eye cannot, for instance, by assigning the result of the test to a grade scale, a ranking, or a description of biological data. Further, the system 10 and unit 12 includes the ability to spatially average over the target medium TM or region of interest 130 within a given spectral subset, and this method and operation can be implemented on a portable device and/or handheld device, as described above.

In a further preferred and non-limiting embodiment, the hyperspectral imaging system 10 and hyperspectral imaging unit 12 can be used in connection with detecting melanoma. As is known, melanoma is a malignant tumor of melanocytes, and diagnosis of melanoma is typically performed by visual inspection of lesions, in order to determine if the lesion is a harmless mole, or a malignant cancer. This visual analysis is done by looking at the asymmetry of the skin lesion, the irregularity of the border of the lesion, color variation of the lesion, diameter of the lesion, as well as change to the lesion over time. Visual analysis of skin lesions for the purpose of identifying melanoma may occur through the use of an RGB color image taken of the skin lesion, which is sent via electronic communication to a dermatologist for inspection. In addition, skin lesion analysis systems are available, which analyze the reflectance of light at distinct, mostly non-overlapping bands of the visible and near-infrared electromagnetic spectrum to aid automated morphological analysis and classification of suspicious skin lesions.

As seen in preferred and non-limiting embodiments in FIGS. 14-16, the person P has a skin lesion that may or may not be melanoma. This skin lesion (and the surrounding skin) represent the target medium TM with the region of interest 130 including the lesion on the target medium TM. In this embodiment, the hyperspectral imaging system 10 and hyperspectral imaging unit 12 is arranged in a manner similar to the preferred and non-limiting embodiment of FIG. 6, including a hyperspectral imaging unit 12 equipped with a lens 14, which is used to take a hyperspectral image and obtain hyperspectral data regarding the skin lesion, i.e., the target medium TM or region of interest 130. The image is taken under illumination of the light source 24, and the hyperspectral imaging unit 12 is connected via a data link 58 to the processor 22, e.g., a computer system. The data link 58 is used for transferring the hyperspectral image data from the unit 12 to the processor 22, as well as for permitting the control of the unit 12. The light source 24 is controlled by the processor 22, such as through the analog or digital interface 60. In addition, the hyperspectral image data may be stored or otherwise used in connection with the database 26, and in this preferred and non-limiting embodiment, this image data may be transferred or transmitted to a different, remote system via a communication interface 132. The hyperspectral image data of the skin lesion may be stored without further processing either within the hyperspectral imaging unit 12 or on an external system, which may be a generic computer, a computer server system on a local network, or a data processing system connected to a wide-area network, e.g., an Internet-based “cloud” service. The hyperspectral image data are analyzed as discussed above, and this analysis can be performed within the hyperspectral imaging unit 12, on the processor 22, on some data processing system external to the unit 12, or a combination thereof. Further, this analysis may be performed in one or more optional phases.

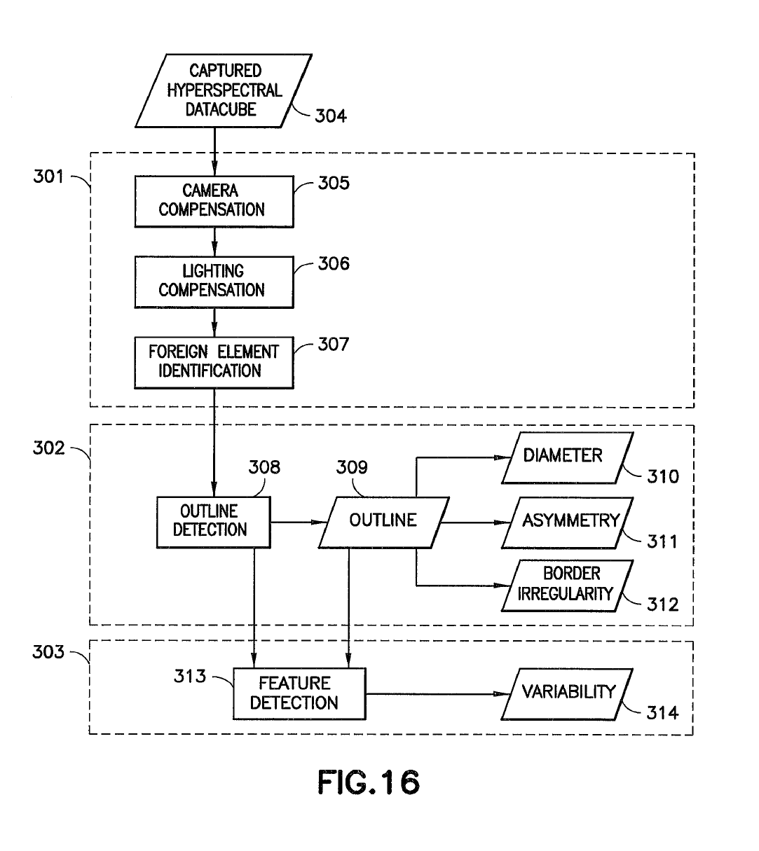

One preferred and non-limiting embodiment of the processing phases for use in connection with the hyperspectral imaging system 10 is illustrated in FIG. 16. In particular, and as illustrated in schematic form in FIG. 16, this preferred and non-limiting embodiment of the process includes a first phase 301, a second phase 302, and a third phase 303. In the first phase 301, image “cleanup” occurs, where image elements, i.e., pixels, are identified and removed that are not relevant to the further analysis of the target medium TM or region of interest 130. In addition, these image elements may be removed from the image data set 304. In addition, in this phase, the process includes compensation of artifacts 305 introduced by the technical characteristics of the specific hyperspectral imaging unit 12 being used, as well as compensation for the spectral profile of the light source 24 being used to illuminate the target medium TM or region of interest 130. Further, and in this first phase 301, the process includes identifying those elements in the images that are not part of the target medium TM or region of interest 130, such as those elements that are not part of the skin lesion, such as body hair. Each of these cleanup processing steps 305, 306, and 307 may be performed in any sequence, or in parallel, depending upon the implementation. In addition, the raw images may be stored (in unedited form) or securely archived to comply with certain regulatory requirements, and to avoid any assertion of tampering. Of course, storage of data may be effected at any step or phase (e.g., an intermediate processing step or phase) of the process implemented using the system 10 and unit 12 of the present invention. This storage function or archival process may be preconfigured for any specific implementations of the system 10 and unit 12, and alternatively, may be configured by the user for any specific implementation or to meet any known regulatory requirements.

In the second phase 302, an outline detection step 308 detects the outline of the region of interest 130 or some specific portion within the region of interest 130, such as the skin lesion. In this phase, the availability of hyperspectral image data, as opposed to conventional monochrome or RGB image data, allows for the use of feature detection algorithms that rely on differences in spectral profiles, as opposed to simple changes in perceived color. Any known hyperspectral feature detection algorithm may be employed, such as those used in earth observation applications, which provide improved accuracy of outline detection based on hyperspectral image data, as opposed to reliance on RGB or multispectral image data. Based on the outline 309 of the region of interest 130 or portion therein, e.g., the skin lesion, a quantification of the diameter 310, the asymmetry 311, and the border irregularity 312 is determined.

With continued reference to this preferred and non-limiting embodiment, and in the third phase 303, further analysis occurs, preferably focusing on the spectral variation within the portion of the region of interest 130. Traditional dermatological examination focuses on color variations of the lesion, which is much coarser. The analysis algorithms used in connection with the described system 10, and unit 12 (and associated methods) rely upon hyperspectral feature detection processes, the accuracy of which is based upon variations of spectral profiles between various image elements. Accordingly, the result of a feature detection step 313 yields a quantification of the variability 314 of the morphological features within the skin lesion. Accordingly, the use of the hyperspectral image data obtained from the system 10 and unit 12 permits the use of improved feature detection methods, which are better than conventional methods that rely solely on apparent color changes in wide spectral bands. The quantifications obtained through the second phase 302 and third phase 303, such as the diameter 310, the asymmetry 311, the border irregularity 312, and the variability 314, are compared against hyperspectral image data, biological data, and/or biological condition information in the database 26. Such comparison against corresponding quantifiers of existing benign moles and malignant skin cancer observed in a wide variety of patients and/or against similar quantifications made of older, pre-existing, irregular benign skin moles of the same person P allows for improved diagnostic techniques.

Any of the phases 301, 302, and 303 described above may be implemented or performed on any suitable device, such as any device or component within the hyperspectral imaging system 10 and hyperspectral imaging unit 12 described herein. Of course, the described process may be implemented or performed on any suitable computing device that is in communication with or otherwise obtains the appropriate hyperspectral image data, biological data, biological condition information, and the like. In addition, this process may include internal and external analysis, for example performing a course initial classification within the hyperspectral imaging unit 12 itself, and a more thorough analysis on the external data processing system, such as processor 22 or any other appropriate computing system, such as a system or unit that has more computational power, additional storage, and memory capabilities and may not always be available on the hyperspectral imaging unit 12 (such as when it is in the form of a portable or handheld device).

As discussed above, and in the various preferred and non-limiting embodiments, the system 10 and unit 12 allow for the continuous or discrete hyperspectral image capture of the target medium TM, the region of interest 130, or any portion thereof. With this image capture, the above-discussed hyperspectral datacube 96 will be generated, which contains hyperspectral data for individual image pixels 98 across a continuous portion of the electromagnetic spectrum. Both the pixel density and the electromagnetic spectrum from which the hyperspectral imaging sensor 18 is capable of sensing may vary according to the component specifications. Further, the diagnosis produced by the system 10 and unit 12 described above may be combined with other sensor data stored in any of devices or components within the system 10 (or third-party systems), as well as the subjective information obtained from the user through a diagnostic aid interface. This additional information may be used in concert with the hyperspectral image data to frame recommendations made for the user based on the findings generated.

As described above, the hyperspectral imaging system 10 and hyperspectral imaging unit 12 according to the present invention allows for the capture, determination, processing, and/or analyzing of hyperspectral image data, biological data, biological condition data, and the like for a wide variety of biological and/or organic materials. Accordingly, and in another preferred and non-limiting embodiment, the hyperspectral imaging system 10 and hyperspectral imaging unit 12 are used for monitoring burns. The American Burn Association (ABA) has classified thermal burns into minor, moderate, and major, largely based upon burn depth and size. The treatment and prognosis of burn victims correlates with this classification. Therefore, it is important that clinicians properly characterize and/or classify the size and severity of their patient's burns. Reassessment of thermal burn size and depth is important, particularly early in the management of patients with severe injuries, as the extent of injury often increases. Accordingly, in another preferred and non-limiting embodiment, the system 10 and unit 12 allow for the identification and characterization of infections that are a serious threat to burn patients. In addition, the system 10 and unit 12 allow for the automated characterization of burns and bruises more accurately, thus allowing for a faster initial assessment and reassessment of bruises and burns. In addition, the burns may be characterized or classified and presented to the user or patient for making clinical decisions.

Common methods are available to evaluate bruises and include direct inspection or visual inspection through RGB images. Such methods are highly subjective and have been found to be inaccurate, as they depend upon the experience of the examiner and also the age of the person, since the ability to see yellow color of old bruises may decline with age. An error rate of up to 50% has been found in controlled experiments. Accordingly, the presently-invented system 10 and unit 12 allow for a more accurate characterization of severity and progression of bruises. When the skin is bruised, the spectrum characteristics of known chromophores, e.g., oxyhemoglobin, deoxyhemoglobin, bilirubin, and the like in the skin, change. The appearance of bilirubin and bruises has been described frequently in literature, and is responsible for the yellowish hue found around the site of a bruise.

As discussed, the system 10 and unit 12 (including the discussed processes and methods for use) provide a comprehensive and accurate hyperspectral imaging system for determining the unique hyperspectral fingerprints, or signatures, known as spectral signatures. Since a hyperspectral image contains a full spectral profile of each pixel, the image can be used to detect and locate the presence and quantity of particular molecules in a sample, thus determining a unique hyperspectral profile for each component of a bruise or burn, e.g., skin depth, oxyhemoglobin, bilirubin, beta carotene, and the like. With respect to this preferred and non-limiting embodiment, i.e., the analysis and/or diagnosis of bruises and/or burns, the target medium TM, the region of interest 130, and/or any portion thereof includes the burned or bruised area. First degree burns affect only the outer layer of the skin, called the epidermis. Second degree burns extend to the second layer of the skin, called the dermis, causing pain, redness, and blisters. Deep second degree burns may progress to third degree burns over the course of several days. Third degree burns involve both layers of the skin and they also damage the underlying bones, muscles, and tendons. The burn site appears pale, charred, or leathery. Fourth degree burns extend through the skin and subcutaneous fat into the underlying muscle and bone. From these characteristics, the burns can be characterized into any of these categories or classifications by looking for the spectral signatures of the epidermis, the dermis, the subcutaneous fat, the muscle, and the bone. Hence, the system 10 and unit 12 according to this embodiment can be used to characterize and differentiate between different types of burns based on their distinct spectral features.

With respect to bruises, the bruising process causes significant structural changes in the skin. When a hemorrhage occurs within the skin, hemoglobin molecules escaping the damaged vessels are considered alien to the body, and the immune system initiates an immediate response to the hemorrhage. By looking for the spectral characteristics of the components of bruises, such as, but not limited to, hemoglobin, oxyhemoglobin, bilirubin, beta carotene, and water, a bruise can be identified and characterized or classified based upon these components, which have spectral characteristics under bruised conditions different from those under normal conditions. For example, it has been shown that the reflectance of bilirubin and beta carotene of normal skin and of bruised skin differ significantly, such that by plotting the reflectance spectrum of these components on the same graph, this important biological information can be determined.

The interaction of light with human tissue has been studied extensively by various researchers and has been used to determine spectral properties of various tissues. The epidermal and dermal layers of human skin constitute a scattering medium that contains several pigments, such as melanin, hemoglobin, bilirubin, and beta carotene. Small changes in the distribution of these pigments induce significant changes in the skin's spectral characteristics. The effects are large enough to enable the inventive process to automatically separate or identify the melanin and hemoglobin in the hyperspectral images. Accordingly, and in this embodiment, the system 10 and unit 12 (and associated methods) can be used to assess, analyze, characterize, and/or classify burns and bruises. In particular, since hyperspectral imaging allows direct monitoring of small ranges of wavelengths, this can result in a more accurate identification of the components found in and around bruises and burns, and lead to a better understanding and characterization of bruises and burns from a simple image or sequence of images or video. It is further envisioned that the process described herein could potentially be used in hospitals or in a consumer setting in order to determine the severity of a bruise or burn, and to determine whether medical treatment is needed. For example, as discussed above, burns are characterized into four categories, and this system and method could make it easier in placing a burn into these categories, as well as assess the progression of infection and the mode of treatment. Accordingly, the system 10 and unit 12 (and associated methods) would be useful to doctor's nurses, and even consumers or patients for tracking how well a bruise or burn is healing, and then alerting the user to when, or even whether, the bruise or burn requires further medical treatment.

In another preferred and non-limiting embodiment, and as described above, the hyperspectral image data can be processed and the hyperspectral image can be overlaid with a grid (e.g., the datacube 96 or a portion thereof), where each square in the grid is a given dimension, for example, using squares of known pixel sizes. The pixels 98 within each square can then be spatially averaged around the center, and the result for each square can then be plotted to provide the spectral information 100 described above, such as a graph with the x-axis as the wavelength, and the y-axis as a percentage. As discussed above, this spectral information 100 (or plot) for each pixel 98 can be matched to a previously-determined spectral signature of a bruise and/or burn, and the bruise and/or burn under evaluation can be characterized based on this matching. Further, and as implemented in one or more of the embodiments of the system 10 and unit 12 of the present invention, the process represents a trade-off of resolution in one or more dimensions for an enhancement in another dimension (e.g., X, Y, wavelength, and/or time), or even indirectly signal-to-noise ratio or exposure time. These represent only a few advantages of this dynamic local conditioning of specified meta-volumes in the four-dimensional time sequence of datacubes.

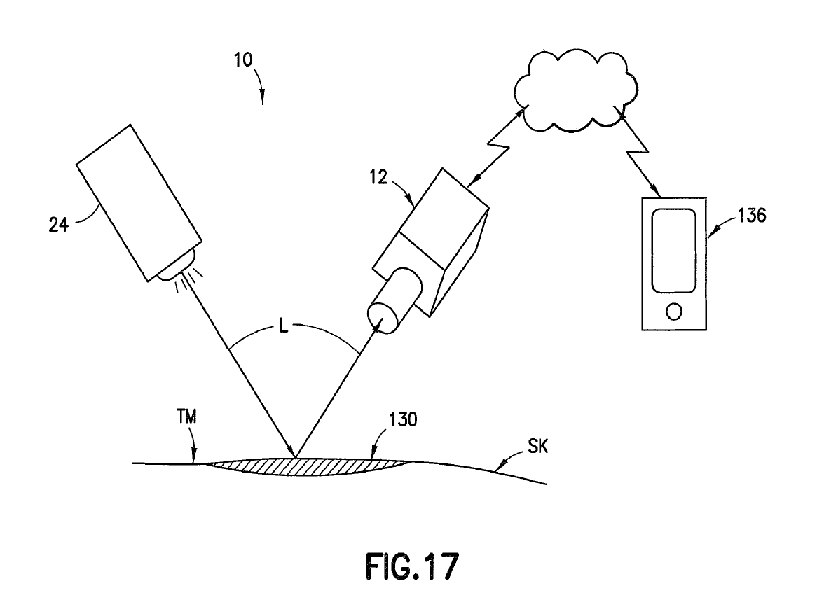

In another preferred and non-limiting embodiment, and as illustrated in FIGS. 17-21, the hyperspectral imaging system 10 and hyperspectral imaging unit 12 can be used in skin hydration analysis. As discussed, hyperspectral imaging can be used to determine many medically relevant indicators of the skin. Accordingly, the system 10 and unit 12 (and associated methods) can be used in analyzing and quantifying hydration of a single region of skin, and a full two-dimensional analysis of a region of skin can also be performed. In addition, an analysis can be performed on captured hyperspectral image data, and the results of such analysis can be superimposed over a standard color RGB image of the skin, whereupon a pseudo-color overlay of hydration level can be presented to the user to show them the two-dimensional hydration level across the skin in the region of interest 130. Accordingly, the system 10 and unit 12 of this embodiment can be used to create an easy-to-understand pseudo-color visualization for assessing general tissue hydration condition, and the results may be combined with other tests to determine skin damage and metabolic state, or for a more complete evaluation of skin conditions.

Surface hydration is of key importance to skin care. Up to 98% of the population in varying degrees has problems with hydration causing issues, such as scaly skin, taut skin, superficial lines in the skin, premature aging, and the like. Given the singular importance of hydration, it is important to understand the level of hydration of the skin in order to treat it properly. Although skin has natural mechanisms for maintaining hydration, the outer layers of skin do not draw moisture from below, as it only receives moisture indirectly when new cells are created below. The outer skin relies on moisture from the outside hydration. Existing methods for quantitatively measuring skin moisture content include bio-impedance analysis, measuring differences in skin impedance, and measuring skin capacitance. In order to determine a remedy for skin dehydration, an analysis of skin hydration must first be performed.

There are numerous types of skin treatments from topical treatments to modifications of the person's environment. However, before treatment of a skin hydration condition, accurate observation measurement of the skin hydration is required. While standard RGB imagery can be used for certain, limited diagnostic functions, in this preferred and non-limiting embodiment, the system 10 and unit 12 (and associated methods) provide a more accurate measurement through the use of hyperspectral images for the classification, diagnosis, and assessment of skin hydration at a single point in time, and over a course of recovery. In addition, the system 10 and unit 12 can be used for a remote diagnosis of skin, to record and transmit images of the skin, and also to compare images over time. Imaging processing can be used for the auto-analysis of skin hydration, and this skin hydration can be measured over a region of interest 130 of the skin to illustrate how hydration varies over this region. The process according to the present invention can also be used to auto-register multiple images of the same region of interest 130 over time. These automatic measurements of skin hydration can be presented in a pseudo-color image superimposed over a standard RGB image of the skin to allow the viewer to see hydration information from the region of interest 130, which is not available to the naked eye. Automatic measurements from the hyperspectral image yields information on a per-pixel basis, which can be displayed overlain on a two-dimensional image of the region of interest 130.

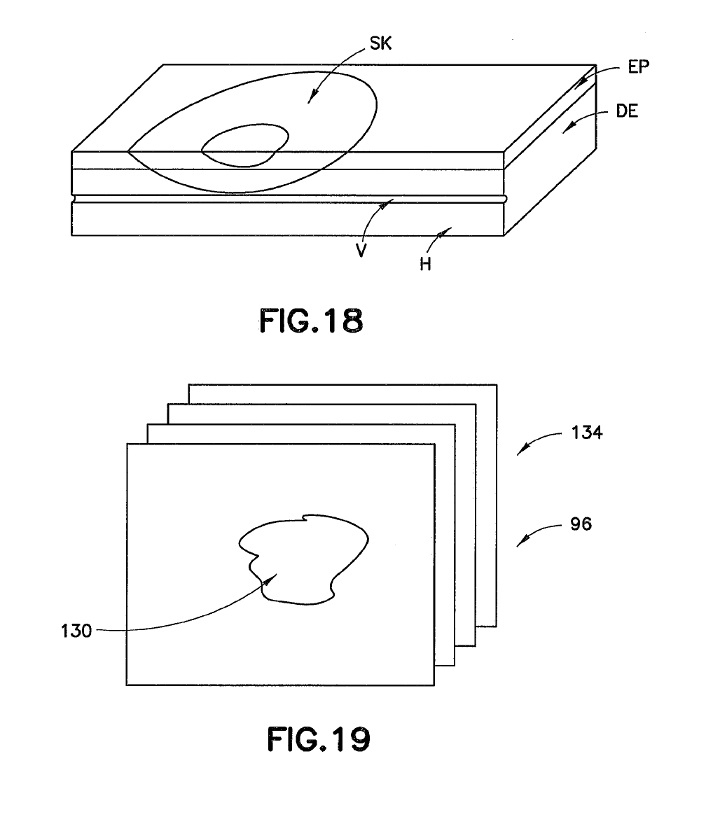

In this preferred and non-limiting embodiment, and as illustrated in FIG. 17, provided is a hyperspectral imaging system 10 and hyperspectral imaging unit 12 (and associated methods) for use in skin hydration analysis. In particular, the system 10 and unit 12 of this embodiment can be used in assessing the skin hydration of a region of interest 130, where the target medium TM is the person's skin. As discussed, and since the hyperspectral image data of each pixel of a hyperspectral image includes a full spectral profile, hyperspectral images can be used to detect and locate the presence and quantity of particular molecules in a sample, thus determining a unique hyperspectral profile for each component of the skin. As shown in schematic form in FIG. 18, the skin SK includes the epidermis EP as well as the dermis DE. In addition, veins V sufficiently near the surface of the skin SK can also be imaged. Below the dermis DE is the subcutaneous tissue or hypodermis H, which may or may not be imaged. Differences in spectrum can be used to determine hydration of the skin SK in a region of interest 130 using the system 10 and unit 12 of FIG. 17.

It should be noted that interstitial fluid reaches all body tissues through the blood, and makes up approximately 70% of the body. It carries with it nutrients and water vital to cellular function. Without this proper exchange of nutrients, water and wastes, cells cease to function and eventually die. Although sufficient water intake is critical in maintaining metabolism, it will not by itself correct existing surface dehydration. The main water reservoir of the skin is located in the two layers of the skin, namely the dermis DE and the hypodermis H. The epidermis EP, the location most vulnerable to fluid deprivation, cannot compensate by drawing moisture from below. Rather the epidermis EP receives moisture indirectly by the production and upward movement of new cells, or by topical moisturizing of the stratum corneum or horny layer, i.e., the outermost layer of the epidermis consisting of dead cells. The purpose of sebum, a hydrophilic fat, is to mix with water from the atmosphere and secretions from the sudoriferous glands to form a hydro-lipid film for the stratum corneum. The constituents of this hydro-lipid film create an ideal surface ecology that is vital for skin health. Further, one of the major functions of the skin is to provide a barrier against moisture infiltration, and because hydrophobic fats in the horny layer (stratum corneum) constitute this barrier, wetting the epidermis EP does not allow moisture into the skin. The stratum corneum, then, prevents transfer in both directions.

Surface dehydration can occur for several reasons. Soap is harsh because it is alkaline, stripping the hydro-lipid film from the surface of the epidermis and leaving the stratum corneum exposed, unprotected, and subject to moisture loss. Skin damage may also result from using harsh chemicals, astringents, or from continual sun exposure. Skin neglect covers a wide area, from failure to drink sufficient amounts of fluid to cigarette smoking, which constricts blood flow in the capillaries, which, in turn, reduces the flow of moisture and nutrition to the cells. Certain illnesses may also cause internal dehydration and ultimately affect the epidermis EP. Diuretics and many cold and flu remedies that dry up mucous have side effects on the surface of the skin. The use of cortisone also induces dehydration. The regular use of scrubs can break down cell cohesion in certain skin types, which reduces the capacity to retain moisture and places capillaries at risk. Climate can also affect skin hydration. Moisture evaporates quickly within the dry atmosphere of air conditioning and/or overheated rooms, and adequate protection should be taken. Friction and heat of hot showers remove sebum from the surface of the skin inviting capillary damage and dehydration. An excessive intake of table salt can have dehydrating effects, as it transfers water from the interior of the cell to the interstitial fluid, creating water retention and bloating at the same time. Coffee, in addition to other negative effects, can also contribute to dehydration.

It is important to first test the hydration of the skin in order to identify the causes of dehydration, and then determine if corrective action is possible in order to determine therapeutic countermeasures, some of which may be potentially lifesaving. There are many potential remedies that can rehydrate skin, including, but not limited to, moisturizers, protective creams, humectants, protection from sun, diet, modifications to climate, and the like. However, an accurate measurement must first be obtained of the current hydration of the skin, and in many cases, the hydration level of the skin may not be obvious to the eye.

As discussed above, existing methodologies are available for assessing skin hydration. The most common methods in quantitatively measuring the skin moisture content in humans are the bio-impedance analysis method and the capacitance method. Bio-impedance analysis is a method of estimating body composition. Some types of body composition commonly measured using this method include body fat and total body water. The basic idea behind the bio-impedance analysis method is that an electrical impedance or opposition to electrical current flow exists through body tissues. A 50 kHz electrical signal is typically applied to the skin, and skin impedance is determined therefrom. This impedance can be used to calculate the water content, i.e., moisture, of the stratum corneum. The capacitance method treats the water content in the skin as a dielectric material. Thus, for a capacitance measuring device, the increase in capacitance is proportional to the quantity of water in the skin. The exact type of transducer used for capacitance measuring devices in relation to skin moisture is called an “Interdigital Capacitor.”

Light incident on human skin may be scattered or reflected by the skin's surface due to differences in the refraction index between the surrounding air and the skin surface. The light transmitted through the air-skin interface may be absorbed by chromophores, e.g., melanin and hemoglobin, in the epidermis and the dermis, or scattered by cells or collagen fibers present throughout the epidermis and dermis. Therefore, the observed skin hyperspectral data are the sum of the surface reflection, also called Fresnel reflection, and diffuse reflectance. Diffuse reflectance corresponds to light that entered the tissue and reemerged from the tissue toward a detector.

By using the presently-invented system 10 and unit 12 (and associated methods), a series of two-dimensional images may be recorded of the biological tissue or target medium TM in the region of interest 130 (or portion thereof) over a spectral band at discrete wavelengths, which can be an image of the skin SK at a region of interest 130 and surrounding area, as illustrated in FIG. 19. The resulting sets of images 134 comprise the above-discussed hyperspectral datacube 96. As discussed, in one preferred and non-limiting embodiment, this datacube 96 is a set of two-dimensional images, each recorded at different discrete spectral bands. Each pixel 98 in the datacube 96 corresponds to the local spectrum of the tissue. Analysis of the datacube 96 can reveal local concentrations of tissue chromophores. The spectra of human skin measured by the hyperspectral imaging system 10 and unit 12 according to the present invention can indicate many things, including, but not limited to, melanin concentration, thickness of the epidermis EP, blood volume and oxygen saturation of the blood in the dermis DE, the scattering properties of the tissue, and the like. As illustrated in FIG. 21, this spectral information 100 can be provided to the user or patient in the form of a graph. In this exemplary embodiment, this graph plots the spectral molar absorption of melanin, oxyhemoglobin, deoxyhemoglobin, and water content (hydration) according to Beer's Law. Water has a peak at around 1,000 nanometers, and by measuring across a region of interest 130 on the skin SK, hydration can be determined. In another preferred and non-limiting embodiment, the hyperspectral imaging system 10 and hyperspectral imaging unit 12 are used to assess and map ultraviolet skin damage.

In one preferred and non-limiting embodiment, and as illustrated in FIG. 17, the hyperspectral imaging system 10 and hyperspectral imaging unit 12 uses the light source 24 to illuminate the region of interest 130 of the target medium TM (e.g., the skin SK), at multiple wavelengths simultaneously. The hyperspectral imaging unit 12 further includes an imaging sensor 18 for capturing images of the region of interest 130 at multiple wavelengths simultaneously, and the image processor 20 is used to aid in the capture of hyperspectral data. In this embodiment, the hyperspectral image data that corresponds to a captured hyperspectral image is wirelessly transmitted to a portable device 136 for further processing. Accordingly, this portable device 136 may include or be in communication with the processor 22.

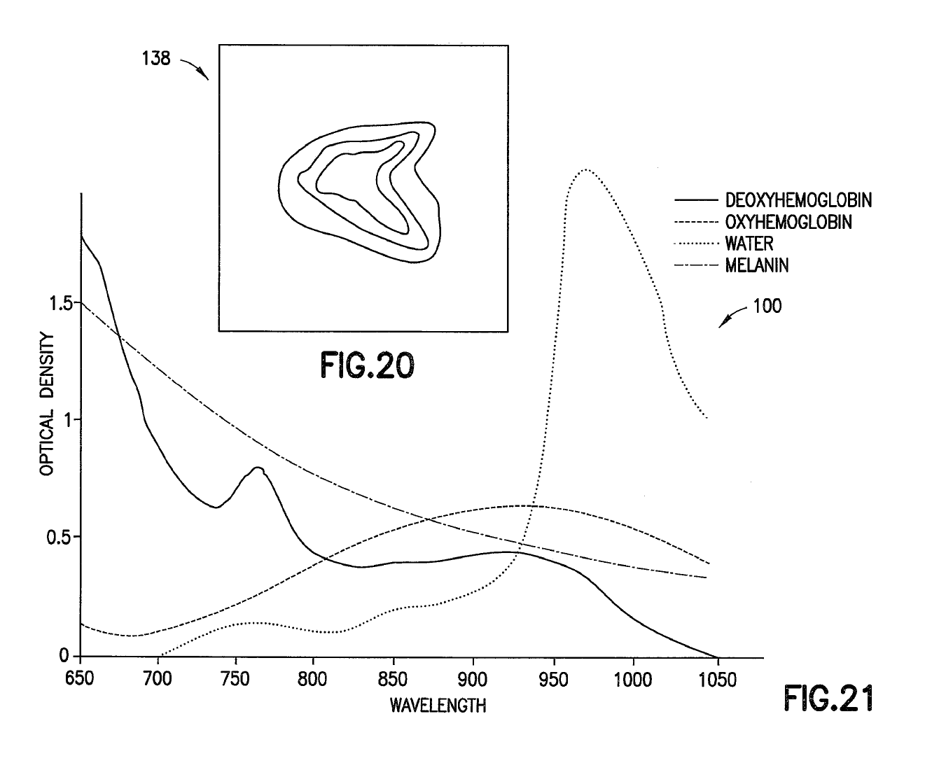

With continued reference to the hyperspectral imaging system 10 and hyperspectral imaging unit 12 of FIG. 17, the multispectral or hyperspectral image data are processed to build a visible image of the region of interest 130 of the tissue, such as by converting the image by performing independent decomposition of each pixel for analysis from which a color or pseudo-color image 138 (in FIG. 20) can be superimposed over a standard RGB image of the skin to provide a better understanding of the level of hydration in the region of interest 130. As discussed, one or more hyperspectral images of the region of interest 130 can be captured in a multispectral or hyperspectral band that includes regions in the infrared, visible, and ultraviolet bands, in order to create a medically-relevant analysis. These hyperspectral images can be captured non-invasively by the hyperspectral imaging unit 12 for use in this hydration analysis.

In addition to analyzing and quantifying the hydration of a single region of the skin, a full two-dimensional analysis of this region of interest 130 can be performed. In addition, analysis may be performed on a captured hyperspectral image, and that information can be superimposed over a standard color RGB image of the skin, whereupon a pseudo-color overlay of hydration level can be displayed on a visual display to the user to show them the two-dimensional hydration level across the skin SK in the region of interest 130. Accordingly, this preferred and non-limiting embodiment of the system 10 and unit 12 allows for the user to better understand skin hydration for cosmetic purposes as well as long term skin care.

In a still further preferred and non-limiting embodiment, provided is a hyperspectral imaging system 10 and hyperspectral imaging unit 12 for the detection and monitoring of acne. Acne vulgaris affects nearly 16% of Americans with prevalence in adolescents and sometimes persisting into adulthood. Aside from physical discomfort, the disease can cause significant psychological complications, such as loss of self esteem, increased stress levels, and in some cases, depression. The disease is caused by blockages in follicles, and affects mostly skin with the densest population of sebaceous follicles. If detected early enough, anti-acne treatments can be applied to minimize the chance of visible breakouts.

In this preferred and non-limiting embodiment, and in order to identify molecular components and characterize the state of acne development, hyperspectral data, such as a hyperspectral datacube, is constructed of the region of interest 130 on the skin (target medium TM) of a patient that is acne-prone or suspected of being acne-prone. In this embodiment, each pixel of each hyperspectral image contains data about light intensity over a wide spectrum, and by previously identifying spectral signatures of various molecular components, one can identify the spectral signatures in a new hyperspectral image by matching, such as by using the database 26. Emerging acne sites can be identified based upon the presence or absence of the spectral signatures. By using this system 10 and unit 12 allows the user to quickly ascertain relevant abundances of oxyhemoglobin, deoxyhemoglobin, and melanin in the skin, which are indicative of emerging skin lesions or acne.

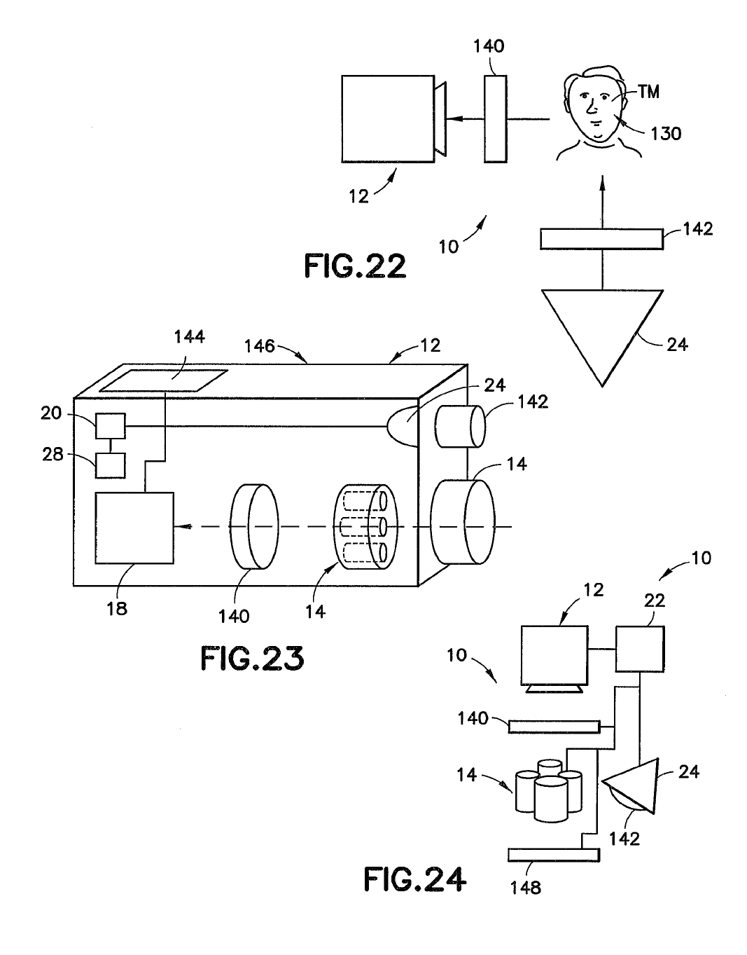

As illustrated in one preferred and non-limiting embodiment in FIG. 22, the hyperspectral imaging unit 12, including the imaging sensor 18, is located behind a linear polarizing filter arrangement 140 (e.g., filter arrangement 16). Further, a diffuse white-light source 24 is located behind another linear polarizing filter arrangement 142, which is oriented orthogonally to the filter arrangement 140. The molecular components of interest are more abundant beneath the surface of the skin. Therefore, light that has traversed some tissue and scattered off the subcutaneous components are of particular interest, as opposed to the specular reflection from the surface of the skin. By using the polarizers (or filter arrangements 140, 142) oriented mutually orthogonally, the amount of specular reflection transmitted to the unit 12 is minimized, and the level of desirable light transmitted to the unit 12 is increased. Accordingly, by using the hyperspectral imaging system 10 and hyperspectral imaging unit 12 according to this embodiment, and processing the resultant hyperspectral data using the biological data, biological condition information, existing or pre-existing hyperspectral data, and the like, such as in database 26, the system 10 and unit 12 are able to locate possible skin lesions related to acne breakouts, as well as emerging acne lesions.

In a further preferred and non-limiting embodiment, provided is a hyperspectral imaging system 10 and hyperspectral imaging unit 12 that are used in characterizing tooth health, where the target medium TM or region of interest 130 is at least a portion of a tooth. In such an embodiment, the biological data are in the form of biological condition information that is determined based upon hyperspectral features of the tooth. Despite major improvements in dental technology, dental caries remains one of the most common chronic diseases of modern society, and is commonly missed until its advanced stages, where it is too late to treat. The initial stages of dental caries are characterized by demineralization of enamel crystals, commonly known as white spots, which are difficult to diagnose. The disease is caused by acidic productions of cariogenic bacteria dissolving the mineral content of enamel or dentin. Calcium and phosphate ions diffuse out of the tooth's surface, resulting in local enamel demineralization. Loss of mineral content is substituted mainly by bacteria and water, which eventually leads to the formation of carious lesions. If detected early enough, such demineralization can be arrested and reversed.

According to the Academy of General Dentistry, there is a relationship between gum (periodontal) disease and health complications, such as stroke and heart disease; therefore, it is important to maintain good oral hygiene. Other research shows that more than 90% of all systemic diseases have oral manifestations, including swollen gums, mouth ulcers, dry mouth, and excessive gum problems. It would, therefore, be highly desirable to catch and understand these oral problems early in order to prevent further complications associated with these oral manifestations. Accordingly, the system 10 and unit 12 of this embodiment can be used or implemented to obtain the appropriate hyperspectral data and images, which contain a full spectral profile of each pixel. This image can be used to detect and locate the presence and quantity of particular molecules in a sample, thereby determining a unique hyperspectral profile for each oral component, e.g., dental caries, plaque, gingivitis, and the like. Further, and as discussed, hyperspectral images, and more specifically, the hyperspectral image data corresponding to captured hyperspectral images, can be processed to gather spectral signatures of the components captured within the image. Further, near-infrared hyperspectral imaging can be used to detect demineralization based on distinct spectral features of healthy and pathological dental tissues. Dental tissues, including, but not limited to, enamel, dentin, calculus, dentin caries, enamel caries, and demineralized areas. By recording specific spectral wavelengths scattered, reflected, or transmitted by the different components within the near-infrared, ultraviolet, and visible spectra, in the teeth and gums, the oral health of the patient can be established depending on the presence of the spectral signatures of specified components.

In one preferred and non-limiting embodiment, and in order to identify oral components to thus characterize oral health, the system 10 and unit 12 allow for the capture of an image or a sequence of images or video based upon these images, the user can then characterize the oral health of the tooth and/or gums based upon the presence or lack of presence of various oral components. In addition, the system 10 and unit 12 can be used to characterize and differentiate between different dental tissues and stages of particular dental diseases based upon their distinct spectral features. Again, all of this biological information, hyperspectral data, biological data, and the like can be populated in the database 26 for comparative and analytical purposes.

In the case of dental caries, the process of forming dental caries causes significant structural changes in teeth by increasing the porosity and water content of the diseased tissue. These changes lead to increased absorption and scattering of the incident light, which can be measured and quantified by the spectroscopic and hyperspectral imaging methods discussed herein. In addition, near-infrared scanning is particularly useful in connection with caries detection, as compared to visible light imaging, as it exhibits low absorption by stain and deeper penetration into the teeth. Analysis of the hyperspectral spectra suggests that light scattering by porous enamel and absorption by water in dentin can be used to quantify the lesion severity. Using near-infrared wavelengths improves light penetration through the enamel, increases image contrast, and can reveal the presence of hidden lesions. The presently-invented method combines this near-infrared hyperspectral with the hyperspectral visible spectrum in order to gather more information about oral disease related to the presence of plaque and gum inflammation.

In this preferred and non-limiting embodiment, the hyperspectral datacube is represented by an image or sequence of images of the teeth and/or gums captured using the unit 12 by capturing all or a portion of the discrete bands within the range of 300 nanometers to 2,500 nanometers. The hyperspectral image data obtained from these images are either processed directly by the unit 12 or the processor 22, where the processing of the hyperspectral image data for each pixel produces for each pixel a plot of wavelength versus amplitude, whether absolute amplitude or normalized amplitude. In addition, the hyperspectral image may be overlaid with a grid, where each square in the grid is given a dimension, for example, using multiple pixels. As discussed, the pixels within each square can then be spatially averaged around the center, and the result can be plotted in a graph where the x-axis is wavelength and the y-axis is percentage. Next, this graph or plot (e.g., spectral information 100) for each pixel in the original hyperspectral image can be matched to a previously-determined spectral signature, and an oral health profile can be determined based on how the plot matches the previously-determined spectral signatures of various oral components. In this manner, the analysis of the oral health of the teeth and/or gums can be provided.

In a still further preferred and non-limiting embodiment, the hyperspectral imaging system 10 and hyperspectral imaging unit 12 is used in analyzing microscopic specimens. Accordingly, in this embodiment, the target medium includes at least one microscopic organism and the biological data are in the form of biological information that is determined based upon the hyperspectral features of this microscopic organism. In biology and medical diagnostics, techniques in microscopy can be used to detect the presence of many organic and inorganic components in a specimen, such as pathogens, biomarkers, fluorophores, and chromophores. Using the system 10 and unit 12 according to the present invention, in conjunction or as integrated with a microscope, provides important information about the chemical and biological composition of a specimen, without the need for traditional bio-marking techniques. In particular, when samples are viewed through a microscope, the magnification causes the velocity of any motion in the sample to appear magnified as well. This can be especially problematic when viewing live samples at high magnification, where, for example, microscopic organisms may be moving quickly, or particles of interest are subject to Brownian motion. Accordingly, the system 10 and unit 12 of this embodiment can be used to study the properties of organic and inorganic compounds of a sample, using the phenomena of fluorescence, phosphorescence, scattering, reflection, and absorption.

In one preferred and non-limiting embodiment, and as illustrated in schematic form in FIG. 23, provided is a hyperspectral imaging system 10 and unit 12, including an imaging sensor 18, one or more light sources 24, a linear polarizing filter arrangement 142, one or more objective lenses 14, a polarizing filter arrangement 140, and a view or display screen 144. In addition, the unit 12 provides connectivity to an internal or remote data-processing device, such as image processor 20 and/or processor 22, including a remote processor. In addition, in this embodiment, the unit 12 is in the form of a portable device or handheld device 146. It is further envisioned that the hyperspectral imaging unit 12 is in a modular and/or customizable form with one or more additional light sources 24 available, such as for dark-field illumination.

As discussed, the further and non-limiting embodiment of the hyperspectral imaging unit 12 of FIG. 23 may take a variety of forms, such as the handheld apparatus illustrated. In addition, the image processor 20 and/or the processor 22 may be integrated with or in communication with various components of the unit 12, and the processor 20 or 22 may include various known components of a computer, including, but not limited to, a battery, a CPU, a computer memory, and the like. In addition, in this embodiment, the communication interface 28 is in the form of a wireless connectivity chip. Of course, the unit 12 may also include the necessary actuators to turn the device on or off, and to otherwise acquire or initiate the capture of images. In addition, it is envisioned that the unit 12 (or any of the hyperspectral imaging units described above) are programmable or configurable. In this embodiment, a researcher may program the unit 12 to take or capture images at predetermined times throughout the day, to record at only selective wavelengths, to adjust exposure time, to adjust the frame rate, and the like. Further, and as discussed above, the hyperspectral imaging unit 12 of this preferred and non-limiting embodiment may be used to capture hyperspectral images of collected samples, with the device on a mount or in a handheld fashion, which allows for the capture of hyperspectral images of live samples, such as skin, hair, tongue, fingernails, and the like. However, and as discussed, the unit 12 can be integrated with or otherwise include various other components of a microscope or microscopic analytical system.

In another preferred and non-limiting embodiment, and as illustrated in FIG. 24, the hyperspectral imaging unit 12 is included with or otherwise integrated with a microscope having a specimen stage 148, additional objective lenses 14 of varying magnification, and a linear polarizer 140. Accordingly, the lens or lenses 14 of the microscope can also be used in connection with or in replacement of the lens system and arrangement of the hyperspectral imaging unit 12. In operation, after a hyperspectral image of the sample is captured, the hyperspectral image data can be uploaded or transmitted to a separate device, such as a remote processor 22 or other computer or computing system. The processor 22 is then capable or configured to analyze the hyperspectral image data and allow access to this image data by the user or other designated users. Accordingly, it should be recognized that the hyperspectral imaging unit 12 may act as a microscope by using the appropriate lenses 14, light source 24, specimen stage 148, and the like in one integral unit. However, and as discussed above, other embodiments of the hyperspectral imaging unit 12 discussed above may be used with or positioned with respect to existing components of a microscope assembly, possibly using one or more components of this existing microscope assembly to obtain hyperspectral image data, such as the light sources 24 or objective lenses 14 of the microscope.

It should be recognized that the various described hyperspectral imaging systems 10, hyperspectral imaging units 12, methodologies and processes associated therewith, and the various arrangements described herein are interchangeable and configurable for a variety of applications. Accordingly, the components and arrangements of one embodiment may be used in connection with the components and arrangements of another embodiment. In addition, the methods and processes described herein, whether for image acquisition, processing, and/or post-processing, can be used in connection with any of the systems 10 and units 12 described above. In addition, the computing systems, computers, processors, data processing, and other units described above can be programmed to implement some or all of the features and functions to determine, generate, capture, process, analyze, or otherwise act upon the various streams of data, including, but not limited to, the hyperspectral image data, the biological data, the biological condition information, any information in the database 26, or any information or data used in the implementation and use of this system 10 and unit 12.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A hyperspectral imaging system, comprising:
at least one hyperspectral imaging unit, including:
(i) at least one lens configured to direct light scattered by, reflected from, or transmitted through at least a portion of at least one target medium to at least one hyperspectral filter arrangement configured to separate the light into a plurality of discrete spectral bands;
(ii) at least one imaging sensor configured to:
(a) receive the plurality of discrete spectral bands from the at least one hyperspectral filter arrangement;
(b) detect light by a plurality of pixels for each of the plurality of spectral bands; and
(c) generate electrical signals based at least in part on at least a portion of the light; and
(iii) at least one image processor in communication with the at least one imaging sensor and configured to generate hyperspectral image data associated with the at least one target medium; and at least one processor configured to determine biological data based at least partially on at least a portion of the hyperspectral image data.

2. The hyperspectral imaging system of claim 1, further comprising at least one light source configured to direct light towards at least a portion of the at least one target medium, the at least one light source comprising at least one of the following: a light emitting diode, a laser, a colored light source, a configurable light source, ambient light, or any combination thereof.

3. The hyperspectral imaging system of claim 1, wherein the hyperspectral image data are in the form of at least one hyperspectral image datacube.

4. The hyperspectral imaging system of claim 3, wherein the at least one hyperspectral image datacube comprises an X-axis, a Y-axis, and a-wavelength axis.

5. The hyperspectral imaging system of claim 1, further comprising at least one hyperspectral database populated with at least one of existing hyperspectral image data and associated biological data.

6. The hyperspectral imaging system of claim 5, wherein the biological data are determined at least in part upon a comparison of at least a portion of the generated hyperspectral image data and at least a portion of the existing hyperspectral image data.

7. The hyperspectral imaging system of claim 1, wherein the at least one hyperspectral imaging unit further comprises at least one communication interface configured to communicate at least a portion of the hyperspectral image data to the at least one processor.

8. The hyperspectral imaging system of claim 1, wherein at least one of the at least one hyperspectral imaging unit and the at least one processor comprises a portable device.

9. The hyperspectral imaging system of claim 8, wherein the portable device is at least one of the following: a cellular telephone, a smartphone, a laptop computer, a pad computer, a handheld computer, a personal digital assistant, a portable electronic device, or any combination thereof.

10. The hyperspectral imaging system of claim 8, wherein the portable device comprises at least one display device configured to display information to a user, and wherein the displayed information is at least partially based upon at least one of the hyperspectral image data and the biological data.

11. The hyperspectral imaging system of claim 1, wherein the biological data at least partially comprise biological condition information relating to at least one of the following: an ear, an outer ear, a middle ear, a nasal cavity, a throat, or any other accessible bodily region.

12. The hyperspectral imaging system of claim 11, wherein the hyperspectral imaging unit further comprises a housing having an end configured for at least partial insertion in at least one of the following: an ear, an outer ear, a middle ear, a nasal cavity, a throat, or any other accessible bodily region.

13. The hyperspectral imaging system of claim 1, wherein the biological data at least partially comprise biological condition information relating to at least one of the following fluids: bodily fluid, blood, urine, saliva, sweat, semen, mucus, or any other bodily fluid.

14. The hyperspectral imaging system of claim 13, wherein the hyperspectral imaging unit further comprises an insertion portion configured to receive at least one of the following: a collector, a test strip, a container, or any combination thereof.

15. The hyperspectral imaging system of claim 13, further comprising at least one test strip configured to contact the fluid, and wherein the at least one test strip is impregnated or coated with at least one chemical that is capable of reacting with the fluid.

16. The hyperspectral imaging system of claim 13, further comprising at least one portable container configured to hold the fluid, such that the hyperspectral imaging unit can be positioned with respect to the container.

17. The hyperspectral imaging system of claim 13, further comprising a testing device, comprising:
a reagent cartridge including at least one reagent pouch containing at least one reagent chemical, the pouch configured to be opened, such that the at least one reagent chemical flows to at least one mixing chamber;
a housing having an opening configured to receive a fluid sample, the opening in fluid communication with the at least one mixing chamber; and
a test strip configured to be positioned in the at least one mixing chamber.

18. The hyperspectral imaging system of claim 17, wherein the testing device further comprises three mixing chambers, including:
a positive mixing chamber containing a positive indicator of a specified biological condition;
a negative mixing chamber containing a negative indicator of a specified biological condition; and
a sample mixing chamber configured to receive the fluid sample.

19. The hyperspectral imaging system of claim 17, wherein the at least one hyperspectral imaging unit further comprises a camera configured to acquire an image of at least one data element positioned on the housing, and wherein the at least one data element is encoded with information associated with the testing device.

20. The hyperspectral imaging system of claim 1, wherein the at least one target medium is at least a portion of a person's face, and wherein the biological data at least partially comprise biological information at least partially determined based upon the hyperspectral features of at least a portion of the person's face.

21. The hyperspectral imaging system of claim 1, wherein the biological data at least partially comprise biological condition information relating to a fungal species.

22. The hyperspectral imaging system of claim 1, wherein the at least one target medium is at least a portion of a tongue, and wherein the biological data at least partially comprise biological condition information at least partially determined based upon the hyperspectral features of at least a portion of the tongue.

23. The hyperspectral imaging system of claim 1, wherein the biological data at least partially comprise biological condition information relating to at least one of the following: a rash, a burn, a lesion, an inflammation, an allergic reaction, acne, a wound, a bruise, a skin condition, a dermatological condition, a symmetric condition, a diametric condition, an irregularity condition, a color condition, a size condition, a depth condition, or any combination thereof.

24. The hyperspectral imaging system of claim 1, wherein the at least one target medium is at least a portion of a tooth, and wherein the biological data at least partially comprise biological condition information at least partially determined based upon the hyperspectral features of at least a portion of the tooth.

25. The hyperspectral imaging system of claim 1, wherein the at least one target medium comprises at least one microscopic organism, and wherein the biological data at least partially comprise biological information at least partially determined based upon the hyperspectral features of the at least one microscopic organism.

26. A hyperspectral unit, comprising:
at least one lens configured to direct light scattered by, reflected from, or transmitted through at least a portion of at least one target medico to at least one hyperspectral filter arrangement configured to separate the light into a plurality of discrete spectral bands;
at least one imaging sensor configured to:
(a) receive the plurality of discrete spectral bands from the at least one hyperspectral filter arrangement;
(b) detect light by a plurality of pixels for each of the plurality of spectral bands; and
(c) generate electrical signals based at least in part on at least a portion of the light; and
at least one image processor in communication with the at least one imaging sensor and configured to generate hyperspectral image data associated with the at least one target medium; and
at least one communication interface configured to communicate at least a portion of the hyperspectral image data to the at least one processor, which is configured to determine biological data based at least partially on at least a portion of the hyperspectral image data.

27. The hyperspectral imaging unit of claim 26, further comprising at least one light source configured to direct light towards at least a portion of the at least one target medium, the at least one light source comprising at least one of the following: a light emitting diode, a laser, a colored light source, a configurable light source, ambient light, or any combination thereof.

28. The hyperspectral imaging unit of claim 26, wherein the hyperspectral image data are in the form of at least one hyperspectral data cube.

29. The hyperspectral imaging unit of claim 26, wherein the hyperspectral imaging unit comprises a portable device, the portable device in the form of at least one of the following: a cellular telephone, a Smartphone, a laptop computer, a pad computer, a handheld computer, a personal digital assistant, a portable electronic device, or any combination thereof.

30. The hyperspectral imaging unit of claim 29, wherein the portable device comprises at least one display device configured to display information to a user, and wherein the displayed information is at least partially based upon at least one of the hyperspectral image data and the biological data.
